SEQUENCE LISTING (68 sequences)

SEQ ID NO: 1 - DNA, 1253 bp, Emiliania huxleyi
ccatcgtgttacgacatccgacctgctttttcacacaaacaacagcgagccgagagccaccgtcggtgagaagccgagcgtgatagaagccgaccgtgtactagccacgaagccacgctcgtcgccggcacggcggcgctctccgtcccgacggccacgcctcgaccgactgccgacgtgggcgtcatcggcgccgggccggcgggactcgtgctcgcccacgtgcttcgcgagcgcggccattcggtccgcatcttcgagcgaatactatcgcgactcgttccgcccggtgggtgctgcggtgttcatgcacccgttcgcgctcaactcgctccgcgccgtctcgcccacgctcgagaagcagctgctcgacgtctgcacgaccatccacacactctcgttcaccactctcgctgaggacgcgccgagcttccggctcgacacgctcggcgacgcgaagcgcgtgctcggcgcgccgttcgtggcggtgcgcttctgggacatgctgcgtgcgttgcgggtcggcctgccggacgagctcttctgctttggccaccagctcgagcggtacgagcagcacggcgacgcggatggtgtgacgctctactttgccgacgccgcgcagccgccgcgcacggtgcgctacctgattgacgcgggcggcatccgctcggcgacgcgtgcgcagctcctcggcgacgagcgcatcccgcggctgcgcgcgaccttctcggtgcttcccgccgagcgcgtcgcggcggcgcacgaggcggtcggcggcgcgtcgacgccgggcgagctgggcttcgtggcgggcgacaacctctcggtggtgacgatgggtctctccgacggtggcgtgatgtggagccagaccgacctcgccgccgacccgacggcgcaactctaccacgacggcagcgacgccgagctgcgcgccgacctcggggccaagtacgcaaagagcaacggctggccgagcggcgtgcgcgcgctcgtcgaggcgacgacgcccgccgacgtcatcgagagcaccgtcgccgagctacccgtggcgtggcggtggggcgacggccacgtgacgctgttgggcgacgcggcgcacgcgcagctgcccgcgctcggcttgggcgtctcgacggcgtttggcgacgtcgaggagctcgtggcgcagctcgaccgccacggcctctcgcagaaggcgctccgctggtacgagcgctgccggatgcccacgtgcgccgcgctacagc

SEQ ID NO: 2 - DNA, 20 bp, Artificial sequence
  Single strand DNA oligonucleotide
gtgatagaagccgaccgtgt

SEQ ID NO: 3 - DNA, 20 bp, Artificial sequence
  Single strand DNA oligonucleotide
tattcgctcgaagatgcgga

SEQ ID NO: 4 - DNA, 793 bp, Emiliania huxleyi
gagagcatcatgcctctgtggcgcctcccggaaggcatcgcaaagttcgacgactggttcggcgcctacccgctcctcgtcttcccggtcaccctctgccccgccctacctgcacgcctgccctcgctgcacgcctgcggctctcgcccctcccgcccgccctctcgcccctccgccccgtcgaccttctcgagagcaccgcgacccgcccggctcccaatcgttacccaccccaccccctcccccgcggcgcccccctccctccccccctccctcccgaccgcaggtgcgcatcttcgaccactcgtcggagacgggcggctcgtccggcctgctcaccccccgcgcggccgagatcgagcagggcgaggcctccgggctgtgggtcgacctcggagcctacggcgtcccgcgcgccgtgcgcgagggcgcggtctgggacgcaaaggagaacatccgcgcgatggagcactggacgcgcgaggtcggaggctggcaggcgctctacaccgacatcttttgcacgccgaccgagctgcgccagatgttcgacttctcgctcctcgacaaggcgcgcaagcggcacgacggcgtcgacgccttcccagaggtgtatgacaaggtcaagtccgaggcgggcatatcggacctctccgccgagctcgccgcagagcgggccgccgccaagaagggcaccgccgccggccgcaggagccagagcgccgccacctccgtcaagtcgcccccccgccgccgcagccgcagccgcagcccccgccccatccggggctag

SEQ ID NO: 5 - DNA, 20 bp, Artificial sequence
  Single strand DNA oligonucleotide
tctgggacgcaaaggagaac

SEQ ID NO: 6 - DNA, 20 bp, Artificial sequence
  Single strand DNA oligonucleotide
catacacctctgggaaggcg

SEQ ID NO: 7 - DNA, 738 bp, Emiliania huxleyi
atgttccactcgacacatgcccgactgctgcccctctcggccgacgttggcgtcattgaggccgtcgccgtttttgtgacggcgacgattgcacttcaaactttcctctttgccaccgccgccgccgtcgagttctcgcgtcgcaggtccgagcgcaaccttggccagatcctgtccgagctgcgaggctcgctctcgaacccgccggcagccatcctgacaacaatggcttgggatcttctcgtgagaccccacaccccgtcagctcagaggtacagcgcgcacgacatgagcctggccagcatgcctcgcatcgaccggctcctgctcgaatggctggccgtctttgtcgcgaccgacttctacatttatttcgagcaccggctgatgcacacgcgcttctggtacacgcggatccacaagcatcaccaccagttccacactccgaccgcttttgccggctttgcccaacaccccctcgaggctgtcctcttcaccgttggctcgctgtggatccaggctatccctccccgtcacgcgtttgtggcaatacaccccttcgctcatgcggcatttggagtgtttggcatcacgtggaccatcctctcgcacgacgatcgctcgtatcacgacggtggcttccactaccttcaccacctgcaccagcaaagcaactttggaggatttacgcacctgtgggactgcctattccgtacacgacgaacaaagcacccctag

SEQ ID NO: 8 - DNA, 975 bp, Emiliania huxleyi
atgcccagccacactccactgctgcaggtcctcctcggacctgcgctcttgcacgcgggcatagcgcccgagacactttccttcgtttttggcagcgagggatccctcctctcggatctgtgcgggcggctcgtctgctggtgcgccttttatgcgctgatccacatcttctaccacatctttgctccgggcgttcgggccttctgcgagaggtggtaccccgacgcaccgacgagtgccgtgccgcagaagctggtcgacaatatgatcgaggtgtcgcacgcggccttcccgctgtacgtgacggtgccgctgctgaccgacttcttccaggtgaagggctggtcgcaggcgtgcggtggcatcgacgactgcggcgggccggcgcgtgcgctgctcggctgcgccgcctactttctgctgctcgagttcatcatcttcgtggaccactactacctgctgcacaagtggagcgtggggaagcggctcggccagcacgccttccaccacgtgtacaagtacgccgaccagctgaacgcgtactctgggtactcgttcgcgccgcaggacggctggtcgcaggggatggctctgccgctcgcgacgctgctggtgcctgtgccgatcgggttcgtgtacctgatggaggtgctcacggggctgtggacgctctacatccacacggacctcttcccgctgccctggcccttcatgggatgcgactaccactacatccaccaccgatacaactggtacaactttggcttcatgactctcctcttcgactccctcttccgcaccgtcaagcacccgcggcacgacgctctcgccctctcgcgcggcgagctgccgatgccaaagctcgacctgctccgctccgcagagctctcctcggccatcctcgcaaagaggggacgtgaggcgctgcagagcgacgacgcgagcgagagcgccgcggcgcgcaaggccgagtag

SEQ ID NO: 9 - DNA, 954 bp, Emiliania huxleyi
atgcccagcactccactgctgcaggtcctcctcggacctgcgctcttgcacgcgggcatcgcgcccgagacactttccttcgtttttggcagcgagggatccctcctctcggatctgtgcgggcggctcgtctgctggtgcgccttttatgcgctgatccacatcttctaccacatctttgctccgggcgttcgggccttctgcgagaggtggtaccccgacgcaccgacgagtgccgtgccgcagaagctggtgtcgcacgcggccttcccgctgtacgtgacggtgccgctgctgaccgacttcttccaggtgaagggctggtcgcaggcgtgcggtggcatcgacgactgcggcgggccggcgcgtgcgctgctcggctgcgccgcctactttctgctgctcgagttcatcatcttcgtggaccactactacctgctgcacaagtggagcgtggggaagcggctcggccagcacgccttccaccacgtgtacaagtacgccgaccagctgaacgcgtactctgggtactcgttcgcgccgcaggacggctggtcgcaggggatggctctgccgctcgcgacgctgctggtgcctgtgccgatcgggttcgtgtacctgatggaggtgctcacggggctgtggacgctctacatccacaccgacctcttcccgctgccctggcccttcatgggatgcgactaccactacatccaccaccgatacaactggtacaactttggcttcatgactctcctcttcgactccctcttccgcaccgtcaagcacccgcggcacgacgctctcgccctctcgcgcggcgagctgccgatgccaaagctcgacctgctccgctccgcagagctctcctcggccatcctcgcaaagaggggacgtgaggcgctgcagagcgacgacgcgagcgagagcgccgcggcgcgcaaggccgagtag

SEQ ID NO: 10 - DNA, 20 bp, Artificial sequence
  Single strand DNA oligonucleotide
ctgctgaccgacttcttcca

SEQ ID NO: 11 - DNA, 20 bp, Artificial sequence
  Single strand DNA oligonucleotide
tcgagcagcagaaagtaggc

SEQ ID NO: 12 - DNA, 738 bp, Emiliania huxleyi; identical to SEQ ID NO: 7

SEQ ID NO: 13 - DNA, 20 bp, Artificial sequence
  Single strand DNA oligonucleotide
gtgtacaagtacgccgacca

SEQ ID NO: 14 - DNA, 20 bp, Artificial sequence
  Single strand DNA oligonucleotide
ggaagaggtccgtgtggatg

SEQ ID NO: 15 - DNA, 1113 bp, Emiliania huxleyi
atgccagacgcaaaggcggccgtgcttctcacagccttctccggcctggcgctgctgatctacgccatccccggcccggtcaagttcggccccatctcggaccgcggcgcccggcccgcctacgccgacaacgcgatgtggcactgcgtgctgtttccgctcgtctttgtgctcggcgcggcggcgggtctctacccgctctcagtgctttacgaccacttcggcgcgacggtcgggctgctcaactttggcgggctcgccttctgcgcgctgctctacgccaagggcctcagccggctcggcctccgcccctccggcccggacgcaggctcgtctggcaaggggctgctcttcgactactactggggcacggagctctacccgcagctgggcggcgtcgacgtgaagcgcttcgtcaactgccgcatctcgatgacctactggatgctcgccggcgtctccttcgccgccgcgcacggccggctcgacccgggcctcgtcctgtgcgccgcgtcgcagtttgtctacctcgtcaagttcttctactgggagatcggctacatgcgctccatcgacatcatcaccgacagggcgggcttctacgagacgtggggctgcctggtctgggtgccctccatctacacgctccatacgcggagcatgcggtcggggtcgcccttaggcgccatcttcgccgtcgggctgctcggcgtcgcattcaacttcgaggcggacctgcagcggcagcgcttccgcgagacgggcggcgcggccctcgtgtggggccgcaagccgatcgccatcgaggccgagtaccacctcgcgcgccacacgagcttgctcctcgtctccggctggtggggcgtcgcgcggcacgcgcagtacctcttcgagctcacggccgagatctgtcaacaggccgcttggtcgtggggcttgctcgccggcgtcggcacgcacggcgcgttgccgctcttctacgcggccttcctcaccgtgctgctcgtgcaccgcgccatccgcgacgaggagaagtgctcgctcaagtacggcgcggcgtacgcaaagtacaaggcgatggtaccgtacaagattgtgccgtacgtcttttga

SEQ ID NO: 16 - DNA, 20 bp, Artificial sequence
  Single strand DNA oligonucleotide
acccgctctcagtgctttac

SEQ ID NO: 17 - DNA, 20 bp, Artificial sequence
  Single strand DNA oligonucleotide
tccgtgccccagtagtagtc

SEQ ID NO: 18 - DNA, 1383 bp, Emiliania huxleyi
atgctccgccccacccgcgcctctcctcggctcgccgggctcaagccggcccggtcgcagtcgccgtcgcgtgccggcggctccacgcccgccacctcgtcgacgaagcccgcacgcaaggctggcggcaagccccagccagagtacgagtttggcggcccagccggcgcggcgggcgtgatgctgctcctgccgctggtcatcggcttcctgtacgcggggtgcggcaagcacgggtgcatacgcctgcccgaggacgcgctcgacgtgctcgccgccgcaccgctcgctccgctgtgggactggacggctgcggcggtggtggctgggtggacggcgctgcacttcctgctgtacgtcgcgctgccggggccgcgggtggagggcgtcgtgctcgcggacggctcgcgcctcaagtacccgatgaacggccacctcgccttttggctctcgctcgccggcttttggcacgggctgccgctgctcggctgcaagcagtcgtgggtgtacgacgcgtacctcccgctcgcgacggcggcgtgcgcgctgtcggtcgccctctccgtctggtcgcacgccctctcctacctcccgaacacacccctcgccgagggaggccagacgggcaacccggtgtacgacttcttcatcgggcggccgctcaacccgcggatcgggtcgctcgacctcaaggcgtgctgcgagctgcggccggggctggtcgggtgggcggtcctcaacttggggctggcggcgaagcagcacgagcagcagggcgaggtgtcttggtcgatggtcgccgtggttggggggcagctcctgtacgtgtgggacgcgctctacaacgagcaggcgatcctcaccaccatggacatcaccactgacggcttcggcttcatgcttgcctttggcgacctcgcttgggtccccttcacctactcgctccaggcgcgcatcctggtggaccacgaccctcgcctccccgcgccggccgtcgccgccatcgccgccctcggcgcgctcggctacgtcgtcttccgcggcgccaactcgcagaaggacgccttccgacgcgacccgtccgaccctgccgtctcgcacctgcagtggatgcagaccaagcgcggcaccaagctgctcgtctcgggctggtggggcgcagcgcgaaagatcaattacaccggcgactggctgatgggcctctcgtggtcgctcgcctgcggcgccgcctcgccggtggcctacttctacccagtctactttgcggtcctgctcgtccaccgcgcgctgcgagacgaccacttctgcgccaccaagtacggcgccgattgggaggcgtacaagcggcgcgtcccgttcgtctttgtgccaggcatcctctga

SEQ ID NO: 19 - DNA, 20 bp, Artificial sequence
  Single strand DNA oligonucleotide
cctctcctacctcccgaaca

SEQ ID NO: 20 - DNA, 20 bp, Artificial sequence
  Single strand DNA oligonucleotide
cgcccgatgaagaagtcgta

SEQ ID NO: 21 - DNA, 3412 bp, Emiliania huxleyi
tccagtggtgtacctgtgttagtgccagtgcgcgccagtcccgtgcccagtgttgtgcgagggagttcccttgtaaattcggaaggctaaaattctctttttgcacaaggaagagccaacgagtggcagagctccccaagctccccaagaagctctcctcctactctaccgaagcagaagcctcgacgctgagatgcccgtgctcgctgacctcctccacgcgctcctcctcccgctccgcatagttctccgcctcatccaccgcctcctctactcgccaacgtggcggacaacctacccggtgacgcgcacgcttcccgagccggtcgcgccggccgcgcgcaagggcacgtggacgctccgtacccgcacgggcgagtcgccgctcgggccggtgaagcacacgctgaacggcaacacgggccgccagctgtggaccttcgacccggccgccgctcccgcggcctgggaggcggacgcgctctcgcactttcggcgcgagtacgaggcgatggccgaggtgcgccaccactcggcggacgagccgctgcggctgcaggcgctgcgcgagacgggcgggaagcggcgcgcggcgaagccgctccccaagggcgccgccgaggcagagcaggtcgacgcgtctctgcggcacggcatcggcttttacggcagcctccagatggaggacggccactggccgggcgactacggcgggccgatgttcctgctgcccgggctggtgatcgcctgctacgtgacggagacggagctgccggcggagcaccgcgctgagatgctgcgctacttgcgcaaccaccagaatgaggacggaggcacggggctgcacatcgagggccactcgacgatgttcgggacgtcgcttaactacgtcgccgcgcggctgctgggcatggcggcggacgacccacactgcctccgcgcgcgcgccttcatgcacggccgcggcggcgccgtcaacaacacgtcgtgggcgaagttctggctcgcgttgctgggcgtgtacgagtggcgcggcatcaaccctctgccgccggagatgtggatcttgccttactggctgatcgtccacccgggccgcttctggtgccactgccgcatggtctacttgccaatgtcgtacctgtacggcaagcgcgccgcctgcccgcccacgcccctcacggaggccctgcgcaaggagctctacgtggagccgtactctgagatcgcgtgggaggagcagcgcaacaactgcggcccggcagacatgtacgtgcagcaccacccgctgcaggacctgctctggaaggtcgtcgccgcagcagagccgtggatccctgcctggctacgccgccgcgcgctgtcgtggtgcgccgacatgatcctgcacgaggacggatcgacacggcatgtcgacatcggcccggtcaacaaggcgatcaacatggtctgctgctggtacgacgacccgcacggggaggccttcaaggcgcacctgccccgcatcttcgactacctctggctcgccgaggacggcatgaagatgcagggctacaacgggtcgcaggcgtgggacacggcctttgcagtgcaggccatcgcggcgtcgggcctcggcgaggaggccgaggtctcgcccatcctgaggaaggcgcacgagtacctcgacgcgtcgcaagtgcgcgaggactgccccgaggtggagaggtactaccgccacatctcaaacggcgcgtggcccttctcgacccaggacccggctgaccgatctcgtcctgctgtatgatatcctgctgcattgtagcttatcattacgataggaccacggctggccgatctcggactgctcttcggaagggctcaagggcgtcatcgctgtcgaggccctcggcttgcccggcgtagcggcgctcgagccggagcggctgttcgattgcgtcaacgtcattctctcgtaccaaaacaccgccgagcactggggcaccggcggctgggccacctacgagaacacgcgcggccccgcggcgctcgagcttctcaacccgtctgagtgcttcggggacatcgtcatcgactactcgtactgcgagctgacctgcgcctgcatcaccgccctctcgcacttccaccgccgccatcccggccaccgcaccgccgagatctcgacggcgatcaagctcggcgcgcaatggatccgcgacaagcagcggcccgacggctcgtggtacggctcgtggggcgtctgcttcacctatggcgcgtggtttggcgtggtcggcctcgcctcggtcggcgagcgggaggcaacctcgccgcacgtcgccaaggcgctcgcgtttctgcgctccaagcagctgcccgacggcggctggggcgagtcctacttgtcgtcctcgaggaaggagtaccaccatgcagacgcgtcgcaggtcgtcaacacgtcgtgggcgatgctcgccctcctcgccggcacggaaggctgcgacgagtcgatggccggcgcggcgccggtcctcgcgcgcgcggctgccttcctgatgtcgatgcaggagccgacgggcgactggccgcagcagctcatctccggcgtcttcaaccacaactgcatgatcacgtacgcaaactaccgcaacatcttccccctctgggccctcggccagtaccgccgccgcctccgcggcgagcccctcccaaacctcgacgcggtccccactctcaagctctgcgagctgccggccggcctgggcgcgcgcgtcgaagtggcgccgcggcggacaggagaacctcgcgccgtgcggcaagagccggcggggcggagacacagcgacagcgaggggtgacgtcgaggtttgcacatcgctggaacaacggcaggccagcgacatgcagaggtagagccgccatgatgggtctcagggagcccctgttttcatgttgaatttattttggcatccgctcgagagagatcgtgtgtggggttcccgaccagctccacaagtccacacccgcaacacctagcccccgcaaccgcacggtgggagcccgccttagcttccctgcagaagagagcatgcgagagttagaccacgcaagcgtgaagcgacaaataagcgagccccccctccccccgccgccgcctggcgccaagccttgtggggtgttgggctcaccttccactgcatcaacacctcccatctagcatacgtacgaattgattgtactcgaggcgtcgaccctcggggcggctcttgcgacttgccgtcgcacttgctcggtggccgggtggaagcagcaagcagccagacctgctatgacgagctcaagtgtgtgggctctctccactcacccttgtccatctcataccttaaaactaccttcaacagaattcggcgaattattggatattaccagt

SEQ ID NO: 22 - DNA, 20 bp, Artificial sequence
  Single strand DNA oligonucleotide
cggagatgtggatcttgcct

SEQ ID NO: 23 - DNA, 20 bp, Artificial sequence
  Single strand DNA oligonucleotide
gcgatctcagagtacggctc

SEQ ID NO: 24 - DNA, 1062 bp, Emiliania huxleyi
atgtcgtccaccttgcagcagaccaccgcgggcaaggttcgccagttcatgggagccgcgagcaagcaggaggcgaagcagggtatcaaggtcgggtccgacatcgaccggtacaacaaccagcacgacctccacggcggcgacgccgaggcgcgcaaggaagggtacgccgacctcgtcaacacgtactacaaccttgcgaccgacttctacgagatcgggtggggccagtccttccactttgcaaacaagtttgcgggcgagaccttcaacgagtccatcacgcgacacgagtactacctggccgcgcggctaggcatccagccgagcgacagcgtgctcgactgcggctgtggcatcggcggccctctccgcaacctcggccgcttcactggcgcgtcgatcaccggagtgactctcaaccagtaccaggtcgaccgcggcaacggcctctgcaccaaggccggcctcgcgccgcgctgcaagctcgtcaaggctgacttccacaagctccccttcccggacaactccttcgaccacgtctactccatcgaggcctgctgccactcgcctgatcgcgcggacgtctaccgcgagatctaccgcgtgctcaagccgggcggctgcttcgtctcgtacgagtggtgcctgaccaagaagcaccgcgacggcgacgcgcagcacatgctctccaagaagctgatcgaggagggggacgggctgcccgacatgatgtcgacgacgcactgcgacgactcgctcgtcaaggttggcttccaggtcgtcgagacgcgagacgcggcgctcgacccgaacccgggcggcgaggcgtggtacgtcatcctcacgccctccttcttcaacctcttccggctgcagttcaccgccatcggcacattcttcatcaacgtcttcatgacgtgcctcgaggcgctccgcctcgcgcccgccggctcgggcaaggtgcgcgagatgctgcggcaggggcagctcggcctcgtcatgggcggcgagatgggcaccttcactcccatgtactacatccacgccaagaaggcctag

SEQ ID NO: 25 - DNA, 20 bp, Artificial sequence
  Single strand DNA oligonucleotide
tcgaccggtacaacaaccag

SEQ ID NO: 26 - DNA, 20 bp, Artificial sequence
  Single strand DNA oligonucleotide
agtactcgtgtcgcgtgatg

SEQ ID NO: 27 - DNA, 747 bp, Emiliania huxleyi
atgggcgcgttcggtgtcatcatcgcgctcgaactgtacgaggagttcaccgcggcgacttactttctcgtctgcggcggcctcgcggcgccgctagtgctgcgcgagttggcgagaggcggcgcggacgggcggccgctgggcgaacggcacggtgcgcgcgcgcagctttggatcgccatctttggcttcatcggcaactactggtacacgcactacttttactgcgtgctgcgcgcgcgctacacggtgcccgcgtggcggctgaatgacgtgcccatctgcatgtacctcgccacccacttttacttttcctcctaccatgtcttcgccaacctggcgctgcggcgcgtcgcgaccggctttgcgcccggcaacctgcgcaccgctctgttcgcgtacgtggtctgcgccctctcgtacgccaccgccttcatggagacgctcacgatctcgcactttccgtactacgactttgaggaccggcacatggcgtacaccctcggctcggccttctacggcatctacttcctcgtctccttcccggtctactacgcactcgacgagcccggcaacacgcctctcgagggcaccggcctccgcggcgtcgccctctcctcgctcggcgccggcatgctggtgctgctgctgctcgacctgacacggctcgccgccgcgggagctccgctcaccatcggaggcaagctgtgggaggtgacgccgaagggactggcggggtccgcctga

SEQ ID NO: 28 - DNA, 747 bp, Emiliania huxleyi
atgggcgcgttcggtgtcatcatcgcgctcgaactgtacgaggagttcaccgcggcgacttactttctcgtctgcggcggcctcgcggcgccgctagtgctgcgcgagttggcgagaggcggcgcggacgggcggccgctgggcgaacggcacggtgcgcgcgcgcagctttggatcgccatctttggcttcatcggcaactactggtacacgcactacttttactgtgtgctgcgcgcgcgctacacggtgcccgcgtggcggctgaatgacgtgcccatctgcatgtacctcgccacccacttttacttttcctcctaccatgtcttcgccaacctggcgctgcggcgcgtcgcgaccggctttgcgcccggcaacctgcgcaccgctctgttcgcgtacgtggtctgcgccctctcgtacgccaccgccttcatggagacgctcacgatctcgcactttccgtactacgactttgaggaccggcacatggcgtacaccctcggctcggccttctacggcatctacttcctcgtctccttcccggtctactacgcactcgacgagcccggcaacacgcctctcgagggcaccggcctccgcggcgtcgccctctcctcgctcggcgccggcatgctggtgctgctgctgctcgacctgacacggctcgccgccgcgggagctccgctcaccatcggaggcaagctgtgggaggtgacgccgaagggactggcggggtccgcctga

SEQ ID NO: 29 - DNA, 20 bp, Artificial sequence
  Single strand DNA oligonucleotide
cctcctaccatgtcttcgcc

SEQ ID NO: 30 - DNA, 20 bp, Artificial sequence
  Single strand DNA oligonucleotide
atgtgccggtcctcaaagtc

SEQ ID NO: 31 - DNA, 738 bp, Emiliania huxleyi; identical to SEQ ID NO: 7

SEQ ID NO: 32 - DNA, 975 bp, Emiliania huxleyi; identical to SEQ ID NO: 8

SEQ ID NO: 33 - DNA, 954 bp, Emiliania huxleyi; identical to SEQ ID NO: 9

SEQ ID NO: 34 - DNA, 20 bp, Artificial sequence
  Single strand DNA oligonucleotide
gtgtacaagtacgccgacca

SEQ ID NO: 35 - DNA, 20 bp, Artificial sequence
  Single strand DNA oligonucleotide
ggaagaggtccgtgtggatg

SEQ ID NO: 36 - DNA, 1955 bp, Emiliania huxleyi
cgcctgcatcctccgcagctgccgccgcgccgcgctcgccgtggcgtgcgcgctgtacgggctgcttcccccgccggcggcgctgccggcccagccgcccctcgcccctccgctcgtgtctggcggcctccctctgctcggccacctcctcgacttcatcaagggcccggtcgggatgatcgacgcgtaccggtcgcggtaccgctcgatgttcacgatcaaggtcggaccgcagcgcatcacctttatgatyggcgccggcccgcagctccagttcatcaaggccaaggacgaggtcctcgaccaggtstttgggaacggcatcgtgtacgactcgccgctcgaggagcggcagcagcaggtcaagctgctcgtgcactcgatgaacacaaaggggctcgaggcgatggtgccaaagatgatcgaygaggcggaggagtactttcgcgagtggggcaacgagggcgaggtcgagatccggaaggtcttctcggagctcatcatcatgaccgcstcgtcgtgcctcatgggccccgagatccgagagaacctctcctccgagatcgcgcgcatctaccacgccctcgacggcggcctcacgccgctctcgacgctgtggcccgccgcccccacccagaagcacaaggagcgcgacgaggcgcgcgccgagatggttgcaatcttctccaagatcattgccaaccggcggagcggcgccgtgcagtcggacgactttctgcaaaagatcatcgactttcggtacaaggacgaggtggacgggagcgggaaggtggtcaaggaggggcgcggcttcaccgactcrgaggtgactgggtggctcatcgtcctcctcttcgcgggkcagcacacctcctccatmaccgccacctggctcggcgccatgctcctctccaacaaagaggcgatggcagactgcctggcggagcagcgggcgcgagtgccggacgaggcgtcgctgacgtacgccaacctgctcgagatggactgcatgcgccgcgccatcaccgagacgctgcgcctctacccgccrctcatcctgctgatgcggcaggtgatgcccgacgccggcctgcaggtgggcgagcacaccatcccgaaaggggacgtggtcggcctctgcgcgcccgcctccaacctcgaccctcgctactggcccgacgcgaccgccttccggccgtcgcgctacctgccggacgcctccaacgargcgatgttcgactcgcgctcggtcggccacgggatgatgcagggcttcatgctctcgtttggcggcggcgcgcacatgtgctccggscgccgcttcggctacctgcaggtgtcgaccatctggtcgatcctgctgcgcgactttgagctgcagatgaccacgccgctgcccaagccggcgtacaacgacatggtcgtcgggccggacgcgcccatcatgatgcggtacaagcgcaaggtcttcctcgcgcccgaagagatcgcggcgaggcaggccaggtacgctgccggcgcggcggccgcaaaggcggagaaggaggcggcggcggcggcggcggtaacggtggtgccgcttgctgcttgccctgaggaggcgcaggcggccctacaggcgcaggcgggacggtacgcggcggcgctggcgcagctcgagcgcgtcaccgccgaacgggacaaggcggtggcggagaatgtcgggctgcgaaagaaggccgagagcctcagcgcgatgctcgcgtcgatgaatgaactggtggaggagctgcgtcgccgcctcgcgggctcactccgtggaggcaagatctcgctcaaatgccgtggggcgccggcctaaattttttagcaccgcatgcacatggacatggacatgtgcatgccgacatgtagcatgtgtgcatatgtgtgcgcgtgtgtttgtgtagcatgctctgggggggcgcgggggcccc

SEQ ID NO: 37 - DNA, 20 bp, Artificial sequence
  Single strand DNA oligonucleotide
gtgactgggtggctcatcgt

SEQ ID NO: 38 - DNA, 20 bp, Artificial sequence
  Single strand DNA oligonucleotide
tccatctcgagcaggttggc

SEQ ID NO: 39 - DNA, 1796 bp, Emiliania huxleyi
gttcgttgcccacgttggtatcacctccttccctgagcctccttcgctcgccgcggcgttcccaaaaggcacatatgcccgacctctcggaccacctcagccccgtgcaggctgctgcgagccgcacgccgcgggcggcggtgccgcgcgtctgtgttgtcactggcggcaccggcttcgtcggcgcgcggctcgtcgagatgctcgttgagcgaggcgctgtggaggtgcgctgcctggacatcgtcccaccgccggccaacgcgtggcgacacgagcgcatcaagtacgtgactggcgacgtctgcgacgaggcgacgctcgatgcgctggttgagggcgctgactgcgtctggcacaacgcggccgcggtcggcccgttccacccgcgggagctgtacttcaaggtcaactacgagggctcgctcaacgtcgtgcgcgcgtgccgcaaggcgggcgtgccaaagattgtcatgtcgtcgtcgccatcaacacggttcgacgggtccgacgtcgacggcctgaccgaggcggagatgccctcgctgccgcaggagtcgtacctgcagctgtacgctgagacaaaggcgatgggcggcacgtgatgccgccgcctctccccgctgcgtcccgcggtagatctggtcgggccatgccgcgtaggcgatgggcgagctcgccgtcacgtctgcgtgctgcgacgagctgatgacgtgcgcggtggcgccgcaccaggtctacggcccgcgcgacaacctcttcctcccaaacatcctcgaggcgacagacacgccgagatgtcgccgagatccgccgagatcggccacgaggtgagctaccgagctctcgccctccagacttgtcgccgagcccctctctcagcccctctctcaggctgcggggacgggccggctgcgcatcttcggcagcggcgagaaccggatctgctgcacgccgaattaaagatcgacaactcacaagaatgaagattgaactaggcgagaaccggatctgcttcacgcacgtcgacaactacgcccacgccctcgtcatcgccgagcgggccctctaccctggctcgcgggctctcggccagttttacatcgtcaccgacggggcgacgcacccgagcccggaggggcacgcgctgctgtggcgggtcatcgacgaggctggcgtcgcgatgggcttcgcctcgctctggcaaaagatgaagctcccgctgctgctgctctggctcgccgcctccatcgcagaggtgctgtcgtgggctctcggcacgcacctcaagctcaaccgcttcaacgtcaaggtgctcaccatgcaccgctggttcgacatctccgcggcgagacatagccagagacatggccagagacatgcccagagagatgcctagagacaccagagactgcgagatgtggccaagacgccggctcgacgtctccgtgccgtcgggcagctgccgtcgggcggtctcgcgcgcctcgtggcgaggggggcgccccttcacacgcgacgccgtgagcaggcgaaccgcgacctcgagtacgagcccatcgtggcgtacgacgtcggctggcccgacacgctgcgctggttccgcgaccactggctgcccaccttcagcgccaagcacagcatgggcttcgctggcatcagcgaccagtcgcaggccaagatcgacgtgcaggcggccggcacgggggcgggccgcccaaaggcggagtaagcagggggggggcgcccgacgtcgccgccctctgaggaagtggtgggtgggtggtggtggtggg

SEQ ID NO: 40 - DNA, 18 bp, Artificial sequence
  Single strand DNA oligonucleotide
gcctggacatcgtcccac

SEQ ID NO: 41 - DNA, 20 bp, Artificial sequence
  Single strand DNA oligonucleotide
accttgaagtacagctcccg

SEQ ID NO: 42 - DNA, 1600 bp, Emiliania huxleyi
acgatcggctcgagatggctgcaggagaggagcccaacgatgaagagcttcagaagagagccgacaactctgacaccgagaaagcagcgctggctgtagactacctagtcgtcggcgccggactcgccagtctagcgttccttgacgagcttctctcgctcgacagcaaggccaccttcgccctcgtcgacaaaaacgaggcacccggcggccactggaattttgcgtacccgttcgtcaggctgcaccaacccgcggcttactacggcgtgtcgagcgagcctctcgcggccggcgcgaagcggagttgcatgatgacgtttgagccgtatgacaacaaggacctcgccgacaaggagaaattgctcgcgtactttgcacgagccgtgcgcaggttcgaggccactggacgggtgcagtactttgcgagggcaaccttcagcgacggcataattgcgtgcgccgacggctccaagcggtcggtcaggatacgccgcaagcttgtgctgcctgcgtccaacgtggtcgtgccggccatgcggccacccgcatatgccgtcgcagagggcgtcgactgcgggcccgccaacatcctctcgtccgacgaggagtcgggccggctgcggacgagctacgtcgtcgttggcgcgggcaagacgggagttgactccgtgcttctgctgctgcggcgcggggtgcctgcgtcggccatcacgtgggtggtctcgcaggactgctggtactttctgcgggactacattttccacagcggcagcgccttcttggggatgagcgtcaagatgctcgacgcgctgaacgagtgctcagactcgtcgagcttcttccacaagatggaggcgatgcggctggttgggaggctcgatccctcgcgaccgccgcccaccacgttccgcggtgctaccgtcgacatcgaggagctagagctgctgcggaccgtcgaagcagcgggaggtgtggtacgcctcggacgcgtcacggccatagacgcgacgggcatcactctgcagcaaggccatgtcgccctgcccagcgccgacacgctcgtcgtggactgcagtgcgaacggcactgacggctacgcttccatccggactcccttcgaggcggaccgcatccacctcaccccctgctactcgttcaacatcggctttggcgccgccatcttggcctggctggagaagaacgtggagaccgacgagcgcaagaacgcgaatgttttcacgatcatcccggaaccgggcgatgtcgtgaagtggggttcccccgcgttgctccccttgattctgtacggccagttcaagatggacaagtcgctcgagcgcctgggcgcgtcctcttggatgctgacgcaccgaacccaccaggcgtcgatgaagcacatctccctgtgcaagcttctctgggccgtgatcgggccgactcgattgatggccaggcgggaccaattcctggcgaagatggaggcaggcggattcaccgacgtgcaattgcataggcgtcacgtcgcgtgagccgtatagggagatcttgtccggggtgtggtgtgcgcgaggcttctctctaaaattgtatataatat

SEQ ID NO: 43 - DNA, 20 bp, Artificial sequence
  Single strand DNA oligonucleotide
gcatgatgacgtttgagccg

SEQ ID NO: 44 - DNA, 19 bp, Artificial sequence
  Single strand DNA oligonucleotide
cacaagcttgcggcgtatc

SEQ ID NO: 45 - DNA, 2501 bp, Emiliania huxleyi
aaaacagacaacccaagggagggatgggaggacggcacgccgccaccacacagggattttaaggagaggagtgtgatgcgctagccgtaggatatccgaacgcctgtgaaaattctcttctgtggcccacgatttcctagcgctcccagcgggccccgatgaagtgcttcaccaaagtgggacgccgggcggccgtcctcctcgaccagcttcgacatcagagctccgtggctcaggcacctggcctccgatcatatcccgtcattggcagtatgctcaacctcccggcagacaacgtgtggcatctgcagcggcgggcggcgaaggcgaacgcgccggacatttatttgatgttcgcaaacggctactccaaaatgggccatttctatacgatggggtttcctggcgtcggagaaggtgtgcacggcaccatctacatctgctccgacccggaggagtacgtgcaggtgatccgaggcgaaggcctgcacccctccggtgcggtcgagacgcagtggccctccatctctgccctcaagcggatgcccggcgcagacgagcccggcggcgccacgcgcggcgcgctaaacatcttcgggcacggcgaggcctggcggcaggagcgcatgttcttccagcaagacctgcagacgccaaaggccgcacgaggatacatcccggggctcgccaaggcggcgcagcttgcgagccggggcgctccggcctgcgccgactcgatggacagcttcacagcccgcagcagcttcgacctcttcaccgccgcattctttgggcagctgatcgagtcggccgaccccgcgacgccatgcctgccgggcaacctcgagtttgcagaggcgatacttgacgcgacgatgctgctcgtgcccatgctgcagagcccagtggagatgctcaaagacagagtcggcgtggagagcgccgatgcccggctgctgcgcgacaagttcagcgtgacgatggagcacgcgtcgggcctcgtgcgctcgtttgtggagcggtgggaggcgggcgagctcaacaagtatgagcaggcctcgtacctccgcgcggcgctcgaccggcggctgaagaacgagcacgtgacggaggaggcgctcgtgggcatctcgttcatcctgctggccgcctccgtcgacacaacctcctcgctggtcaactggaccctgcttcagctggcggtcgacagggaggcgcagcgcaagcttgccgccgagctgcagcgcgagcttggcgtcggaggcagtctgacgccagaggtcacgcagagtggccgcggccagtggctgccgtaccttcggtcggtggtgcgggaggtgcaccggatgcgaccttcgatccccgtgagcctcatgaagcggaccgccgccgaggtcgacatgtgcggctacaccgtgcccgccgggagcctcgtcatgctcgacagcttctcgccgcagaatgacccggcgctggtgccggacgcgcacgagttccgcccggagcggtggctccccgacgcggttgtggcgaggaaggggacggcggcggaagtcatcgaccacccgctgttgcgcgcgcccttctcggcgggcgcccggatgtgccccggcaacaagattgcgaacttcgagctgctgacgatggtggcgcagctggtgcaggactgggagtttgagctcgaggacgcgtcgatcacgagcctgctcgatatcccgtaccaccaaggcaccaccgtccagccgcagccgatgccgaagttcgtcttccggaagcggcgacccgcaacggcctgaccaggaagtgcttcttggcggctcgcgtgtacgtgtggatgggagaggattcccacctcaattgcctttctcgacctctgtcccggcgcgccgctgcgttggcgccgtccgtccggttgcgcgctcttgatgaaggttcataagtgcccgtctccgcataagggagaagcttccttaccttacgtactgagtctgcaataaataaataaatcacacggacacctcgagctactgggagatgggtaaaaaggagcaagtccccgccctgcgaagtgcgatctttgtgcggggttctttgtgggtgtggtcggcgcgactagcctattctcgcacagctgtcctgtaaacaagattacattttgcgccgcgtggtctcgtaacaggcatgtggctctccagtcgaaacttcatcgccccgcgctggcggcggcacacatcacgcaaaacaaacgctgtctaggacgagcctaatccgccgcccacgccgccatcgccgccacgatcgcctcgaacgacacctcgccgccgccctcgccgcctccgccaagcccgccaccgagccctcccccccccccccctcgaagcctatggcgtagacgcacggatcgccattcccgtcgtccttgcccccgaccttgattgagaatggccgcgacgcatgcgatcgaatagagtggcgccgaaaaaggcccaatctctctctgg

SEQ ID NO: 46 - DNA, 20 bp, Artificial sequence
  Single strand DNA oligonucleotide
gctcgatatcccgtaccacc

SEQ ID NO: 47 - DNA, 20 bp, Artificial sequence
  Single strand DNA oligonucleotide
cgggacagaggtcgagaaag

SEQ ID NO: 48 - DNA, 4016 bp, Emiliania huxleyi
gaaaacagacaacccagggagggatgggaggacggcacgccgccaccacacagggattttaaggagaggggtgtggtgcgctagccgtcgcccacccttgggcgtctgcttcttcctatctctcttcgcagcccgtcgccgcttttcgaagtctttctttcgcacacgcggctcacctgcttgcacacactcaacaaccttgaacacacatgctccgccccacccgcgcctctcctcggctcgccgggctcaagccggcccggtcgcagtcgccgtcgcgtgccggcggctccacgcccgccacctcgtcgacgaagcccgcacgcaaggctggcggcaagccccagccagagtacgagtttggcggcccagccggcgcggcgggcgtgatgctgctcctgccgctggtcatcggcttcctgtacgcggggtgcggcaagcacgggtgcatacgcctgcccgaggacgcgctcgacgtgctcgccgccgcaccgctcgctccgctgtgggactggacggctgcggcggtggtggctgggtggacggcgctgcacttcctgctgtacgtcgcgctgccggggccgcgggtggagggcgtcgtgctcgcggacggctcgcgcctcaagtacccgatgaacggccacctcgccttttggctctcgctcgccggcttttggcacgggctgccgctgctcggctgcaagcagtcgtgggtgtacgacgcgtacctcccgctcgcgacggcggcgtgcgcgctgtcggtcgccctctccgtctggtcgcacgccctctcctacctcccgaacacacccctcgccgagggaggccagacgggcaacccggtccgagcagagcctccgccccgcctcggatccacctcggatccacctcggccaaacctcggcagcacctcggccgcaccacgccagcaccacgccagcacctcgcaggtgtacgacttcttcatcgggcggccgctcaacccgcggatcgggtcgctcgacctcaaggcgcgcacacacctcgcccacacctcgcacacacctcacacactcgcacacacttcgcacacacctcgcacacacctcgcccacacctcgcccacacctcacacactcgcacacacccctcgcacacacctcgcccacacctcgcccacacctcgcccacacctcacacactcgcacacacccctcgcacacacctcgcccacacctcgcccacacctcgcccacacctcgcccacacctcccacacacctcccacacccctcgcacacacctcgcccacacctcgcccacacctcccacacacctcgcccgcacctcgctcgcacctcccacacctcggctcgcctacgtgcctcccaggcgtgctgcgagctgcggccggggctggtcgggtgggcggtcctcaacttggggctggcggcgaagcagcacgagcagcagggcgaggtgtcttggtcgatggctgattatacaacgagccagaggttgctaatgaactcgtaatcaactaggcgaggtgtcttggtcgatggtcgccgtggttggggggcagctcctgtacgtgtgggacgcgctctacaacgagcaggccagctctcgagattggccggagattggccggagattggccggagattggccgggcgagatgactcgctcttagaacctagcagcgtgatctcgcgtgatctcgcgtgatctcgcgtgatctcgcgtggcctcaccctcagcgatgagtgtgactctgatgtgactcgcaggcgatcctcaccaccatggacatcaccactgacggcttcggcttcatgcttgcctttggcgagccccgcgcggcctgccacctccctccacctcccctccttcagccgtcgccggggaatggcccctcaggcgacctcgcttgggtccccttcacctactcgctccaggcgcgagatatggggagatatggggagatatggggagatagagctgggtccccttcacctactcgctccaggcgcgcagcctcgtcggcccctcgcccgtccgatgaatcagccgcccacccccatccatccaccaggcgcgcatcctggtggaccacgaccctcgcctccccgcgccggccgtcgccgccatcgccgccctcggcgcgctcggctacgtcgtcttccgcggcgagctgagacggagccgagatgcgagattgcacgagatttgccaaagcacgacttccgagccggactgaagggggcgggggggggggggggcatgggggagggagaggcgagctctgagatcgtccgagatcgtccgaggcgcgcgcagaagagtttcagagctgctgacaatgacaggcgccaactcgcagaaggacgccttccgacgcgacccgtccgaccctgccgtctcgcacctgcagtggatgcagaccaagcgcggccccgagccgctcgtcccggcggctgcgaggcccgttctcggaccgccgccttctcgccgctgataggcgcggcaccaagctgctcgtctcgggctggtggggcgcagcgcgaaagatcaattacaccggcgactggctgatgggcctctcgtggtcgctcgcctgcggcgccgcctcgccggtggcctacttctacccagtctactttgcggtcctgctcgtccaccgcgcgctgcgagacgaccacttctgcgccaccaagtacggcgccgattgggaggcgtacaagcggcgcgtcccgttcgtctttgtgccaggcatcctctgagtagtcctcggaggatcccgccgaggcgcgaccgccggcgaggcatgctgtatgcccggcgcggcagagggcagagcccgtgccgccgggtcatatcctcatatccgaggcagacgccgcgcacaatcattcgcgttccgatgaccgatgtgaggacgctcagcataggagatcagtaattacgcgggtacccagcgtcacgctcgagcgctacaacgcattacgattacgcgtgctgtggccttgctaggagctcgcgctcagcggcgcctttgcacttgaactcttgagaagactctcttacatatgtataaaagtgaaatcgacgatcgcatgcgaagcgttgcgcgactcctagcgttatgcgagggggggcgccgcgctgcacacagacacacatcacaaactcaggacacaggagcgcatgggacggtgcagggctcgctctcctctcgctctccgctgccacacatatgcatgtcacatgtgcagcaccatggacgtgccgttcgtacctctacctgccctgcgcatactcgatgatcccggatcctagatccaatacattagcttccgccactctcgggcctctaaattaacttcggtgccagcgcgccagccagcgctgccctcggtgtgccagctcagacggtagtgcccgtcttcgtcaactttacttgatccatagtcgcagccgaggacgtctgcagtgcacagcgtgacggcggcgacgcgggagaagccaaaggaccccccgtcctcctcctcgtcttcaatgtaattgtaccaaaaggccccgttctcctcgtgcgtctcattctcgctgctcgtccgtcgcagcacctcgtcgcgccgcccgcacgcaccgagcgcgatggtgccgtcgggtgccctcgctccaacaaagatgtgcgtcgcctccgccggcacgctctcgattttcataagagtcgttacgtgggagtacggctcatcgatgtgctgcttccatccggtgagaaaggcatttggaatcgaccgatgcacgccatgagacatgcgcggccgagtgcgcgggctcgaggcagacgcagctgcggaggcggtaggccgcagcgcctggtggcggtacgcctcgtagagcagctccttgccgccctcgtacgcggtcagcacgccctcgacgcggtcggcgaggaaggttggctccatcagcgggtagcgcacgagagagaacagctcgagcttctgggcggcggcgaggggcggcgag

SEQ ID NO: 49 - DNA, 20 bp, Artificial sequence
  Single strand DNA oligonucleotide
tctttgtgccaggcatcctc

SEQ ID NO: 50 - DNA, 20 bp, Artificial sequence
  Single strand DNA oligonucleotide
tctcctatgctgagcgtcct

SEQ ID NO: 51 - DNA, 1814 bp, Emiliania huxleyi
gggcagcgacgagcggcgcgacgcgacgctgcggtacgagcgcgaggctcgccgctacgacctcaccaaccgcaaccgctcgagcgcggccgccgccgcaaacaagcagggcgggtacggcaaggtgccgcagcactcggcctcgaaggtgcggcagatggcgcgggtcgacgagctcttctccgccgtctggctcaagtttggctcgcgcgccctctcctccaacctcgtcgtgccaaaggcgggcgcggcgggcgcggcggcgggcgcggcgagcgacttggtcttttgcgaccggatcttggggctcgtctcgcggtcgttcgcggcggtgatccggcagctgcccgcggagatggtgctcgacgtgctcctcttctacctcgtgctgcgcgcgctcgacaccatcgaagacgacatggaggcgttcaaggacgacgaggcggcgaagtgcaggcacctcaccgccttcgccgactcgtacctcggcgacgaggcgtggtcgctcgacggcgtcggcgagggcgcggagcgggagctgctgcagcgcttcggcgccgtctcgcgcctcttcaaccggctgcccgccgagtcgcgcgaggtgatccgcgacatcacccgccggatgggcgcgggcatggcggagcaggtcggggtcgatctcgggcagggcaccgccgacctggccatgtacaaccggtactgccacgcggtcgccggcctggtcggcgaggggctgacgcgctcctttgtcgcgcgcggcttcgagtcggtctcgatcgccggccaggggaggctcacgtggccgttttgccagccggcggaggaggcggcgggcgggcaaactacgggctggcaaactcgatgggcctcttcctgcaaaagacaaacatcttgcgcgactacctcgaggactacgtcgacggccgcgccttttggccgcagtccgtctggcgcgagtttgcgcgcacgtccgacctcggcgagtttgcgaggccgacggcgcatggcggcgggctgtacgcctcggccttcgacgccgccgccgacccgcagggcggggcggtgatcgggaagggcacgcgcacgtccgcgctcgcgtgcctcaacttcctcgtcgccgacgcgctcgagctcgtgcccgactcgctcgagtacctgcggcggctgcggacgccggaggtcttccgcttctccgccatcccgcaggtgatggcgatcgcgacgctcgaggcgtgcttcgacaacccgcaggtcttctcgggcgtggtcaagatccgcaaggggcaggccgcgcagctcatcctcgcgtgcggaaccatgcccgccgtcgaggcttggttccacgactttgccacgcgcatcaagcgccgcacgcccgccgacgacccgtcgcgaccaaagatcctcgcggcggcagaggaggtgatccgcctcacggcggcctccgccgccgagcagcgccagcgcgcggcggtgcgcaatgcgctgcccgccgccgcggtcgcggtcgcggttgcagctttggccactcgctacctcggcgtctttgccggcgcctaaagccgttagcggcgcggcgtcgtgggtcatgttgctgatagcgcggttactcttccggtcatgggggaagggggggggggggggggcgagagagagagagagagagagagagagagagagagagagagagagagagagagagaggctgcgagagtgccatggtgcattatttttggcgcggaggccacgcacacccacacgtgcgcacccagtgtactgtgtgtagtttcgcataaagcccccgtaagcag

SEQ ID NO: 52 - DNA, 17 bp, Artificial sequence
  Single strand DNA oligonucleotide
cggcgcctaaagccgtt

SEQ ID NO: 53 - DNA, 20 bp, Artificial sequence
  Single strand DNA oligonucleotide
ccgcgccaaaaataatgcac

SEQ ID NO: 54 - DNA, 2048 bp, Emiliania huxleyi
acgaaccactcctgcaacgacacgctgaagtcgacctatgatcgtcccgcagcagccatcctggcagctcgcagtctcgcgccgtctcggcctcgtgtgcgccagcctcgcgctcatcgtgctcgtcgcgcaggcttcgccgccttctgaggtgtctgacgcctctgagcagctggaggagcgggtgccatcgtgctatccgttctgcggaatggaagtggtcgtgcacaagggctacgggctcgagcgccactacgcgaccacgcccgacgggtacatactcggcctcttccgcctgcttcctcgtggcgagaagtccgccgctcggctcgcccggagcccgccgctggtgctgcagcacgccctgctcgattcatcctttgcctgggtatccaactaccacaagaaccagtcgctcgccctcctcgccagcgatgccggctacgacgtctggctcggcaacaatcgcgggaacgtctactcgcgcaaccacacccgcctcgaccccgacgccgacgcggcgttctgggactttacctttgaagacatggcgacggcagacctgcccgccacactgcgctacgtcctcgagcaccgcgctgcggaggcagcggccgcaggcggcggaggaggaggcggcggcggtggtgctcgcggcggcgatggtggcagcggtggtggtggcagcggcggcggcggtgatgatggcggtggcggcggcggggatggtggtggcggcggcggcaagggcagcgtctctacaaggcttcggctgcccgcgaggtcgattggcttcatcggccactcggaagggacgatgcagggcttcgcgggcttctcgctcgctgagaacgccgaggtggcggaggcagtcgctctcttcggcgccctcgcgccggtcgctttcctctccgggatcgactcgcccgtggtgagcggaatggcggcgaaccacgtggacggcctcgcttcgtggctcggcctgaccgagctgctgcccggctcgcgcctcaacggcttctcgtcgccgctgtgcagctgggtgccgctgctgtgcgacggcgtgctcgacttggtgaccggggcgagtgacaacctcaacctgacccacctgcccgtgcagctggcgcacacgcccgccggcacgtcggccaagaacctggcccacttcatgcagggcatccgcgacggccactttcgccggttcgactatggcgacgaggcggagaacgcgcggcggtacaacgcgacgcggccgccggacttcccgcttgcggcgggcttcaaggtgcccgtcgcgatgtggtcgggcgcgcgcgactcgctcgccactccggcggacgtggcgcggctgcaagaggcgctcgcgccgacggggagggtggtgcaccacgcggaggtcgatttcgcgcatctcgactttacttggtcgacacgcgccgcggaggcggtctaccagccgctcctctgcatgatgcgcgcacggctaccgcccgagagagggccgtgcgccgccgaggctgcagccgagcccacacccggccttgcagccgagcctgcggccgaaagacggcgcagcggcgtcttgcaatgagaaagtgcgaaggtctcgcgcggcagctcgctcgctcgaggccggctccttcctcccttcctagcagcctaggattcggttcgcctcttccccatccaagtccccatctccctctcgctctcgcccatcgcgagaatggggtccccacagtagagtagttcatgcgcatctgggtgtgttacatgcacagcatacagtgtccagcctgtcagattcgcgcgtcgtgtcggtcgagagcccctattcttgcgcccatttattttcgcgagcctgtggtatctgtctactaacgggctgagcggtatgcggtatgccgcgcccgcggcaaatgccgcgcccgcggcaaagagtgagggctcgacggagtgcgagtgggcggagagacagagagcgtccgagaccgagtcagaggtgacagcactgacaggtaaggtaaggtaaggtaaacgtctcc

SEQ ID NO: 55 - DNA, 16 bp, Artificial sequence
  Single strand DNA oligonucleotide
cccacacccggccttg

SEQ ID NO: 56 - DNA, 20 bp, Artificial sequence
  Single strand DNA oligonucleotide
gagggagatggggacttgga

SEQ ID NO: 57 - DNA, 1001 bp, Emiliania huxleyi
atcattgtcgctagcgggcgtcccgcaaaaaataagaaaaacgcgcgtcgagagatcaccgaggacggccgatctatcaagtgcgtctagtacgtgtgtatatgtccgactcgcatccagccagaggcgcgatcgccgcagccgttctcctgtaccttctgcacgccctctgcttctccggcaagttgtggcaatggaacgagcaagcctgggctgggaacgagcgcctccgctttgcgttgatttcgagtctgttcctggaggttacgtactggttggcgcagctactcaactggctcattatgcgggatgcttccctgtaccagcgctgggccatagcagagcgaggtgaccgtgtcccccccgctcccgagctcctccgaaagtgcctcacggacctttcggtgagccactgcgtgaggcctgtgctcctatacgctgcttactccatctaccttgcacgcggagcgccaagtagcgcggctcttataccaacggccaccgagctcgtcgctcatttggcctttgcgatcgctgtcgatgacaccatgttttattgggcgcatcgggcgctgcaccatccttgcgtctataagcacatccacaagcagcaccacgaattcaagcagccggtcggtctcgcaaccgagtacgcacacccgctcgaggaagcgtgcaacacgctcgccaccgcgctcggcccgctcctcttgggctcgcacgtggctgtctcggtcggctacatgggcctcaaactctggcagtcgattgacgcccactcggggatgctactcccggtaccactctcaccgtggaacctcttgccggggatggactgcgcggccgctcatgatttccatcactcgcacaacgtgggaaattttggcggcttcttcacgttctgggaccgcgtctgcggcacggacgctgcataccgtcggttcctcgagaagaagaagcagaggtgatgcgagcgagtcatcaaaacgagccagcctcta

SEQ ID NO: 58 - DNA, 20 bp, Artificial sequence
  Single strand DNA oligonucleotide
cactctcaccgtggaacctc

SEQ ID NO: 59 - DNA, 20 bp, Artificial sequence
  Single strand DNA oligonucleotide
aggctggctcgttttgatga

SEQ ID NO: 60 - DNA, 1769 bp, Emiliania huxleyi
gaaaggacttgggcatcaatgcccagcaagcagcccctcggcccgtcgccgcggtccgtctaccgccgcgcacgcgcgctgcacgccgcttgggacatcgcccacggtctcggcgtgtgcgcggtctcgctctacctccttgcgcggcagcgcgaccccgcccttgtcggcggcgggtgcgagccgctgcagtgccgcgtcggcgccgtctggcgcgcggcgtccgccgacccgctcgaggcggcctgcgcctgcttcaccaactttttcttcgtctcgatgtgcggcaactcgcccgccgcgtgggcggcggcgctctggttcgcgagcgtggacgaggtggcgtggggcgcgtttgcggcgggggtggcggcgcgctggggcgacctgtacgacggcggcgccggctacgcgtacggcgtctgcaccttcctcttcttccttgtgccgtacctcctccacggcgccctcctcctccctctcgagctctggagcccggccgtcgccgcagggactccgtacaagctccagccgcagaggagggtcgacgctggccgcatcccgcgcgtggtggccgtgtctgtgctcgagctgtgcttggtcgggctgccctacgtgctcgccctcgcccacctctccgtcgcatcgcgcggcgagcgcggcgtgaggctggagggggcccttcccagctacgcggagcgcgcgtggatgctgctcgctcatctgttggtcaacgaggtgctcttcttctacgcccactgggcgctgcaccagggccccctctaccgccgcatccacaagatccaccacgagttcaccgcgccgtttgcactagccgccgtgcacgcccacccgctcgagctcctcacggcggacttagtccccttcaccgccggcttcgtcctcttccggcctcacatcttctttgtctttttgtggatcgtcggcgctgccctcggcacccagacgcaccactcgggctaccgcctcccctggatcgcagccttcgacgagcagccagacttccacgactttcaccaccagcgcttcagctgctgctacggcaacatcggctggctcgactcgctgcacggcacatcgaggccgtaccgcgaggctcaggcggcggccgaagcgaggcgagacaaggcgcaggaccggtgggaggaggagcgggaagagcgggaggagcgggaggagcgggaggcgaggcgggggcagcggtgccgtgcgtccgccaccaaggccgccgaggcggaggtggaggaggagggggaggaggaggcgggggcggcggcggcgggcgccaagcggcgggccgccgcgcgcgtcgcgcggcctcgcaactggccgctctccacggcgctcagcacggcggtcgggctgcactcgccgctcgagcatggctgagttctggactcggcgggtggctgtgtggcagggtgcgcgcgggcgcgccggcagctgccggcagacccagacggcgcagccgcgtctgatcatgcgggacgaagacgccacggggtggtgggggggtggcgggtgcattgtaggatgtaggagtaggtctggctgattgactgataggttcataggaaggtccctgtcgcgctatctctagcgcaagggtcgagggagggggcgagtttgagaactctacatctactgtttwaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagg

SEQ ID NO: 61 - DNA, 18 bp, Artificial sequence
  Single strand DNA oligonucleotide
gagttctggactcggcgg

SEQ ID NO: 62 - DNA, 22 bp, Artificial sequence
  Single strand DNA oligonucleotide
gacagggaccttcctatgaacc

SEQ ID NO: 63 - DNA, 816 bp, Emiliania huxleyi
atgtggagctattctgtcctcctccactacggcgtcggcacgtacgccgtcctcctcggctcctacctcgccgcctgcctcccatttgccgtcctcgaccgctccggcttcgcgacgcggtggcgcatccagccgagcccgcctcgacaggacgtcgcggctgcgaagcgcatggtcgtgctcaactttgcgtggctgctcccggccgtcctcgccgcgtcgccgctgctccggctcctcttcccctcggacgctcagccgccgccgctggcgagcgctccgttcacggcgttggcgtggttcgtgctgcacgacctctccttctattgctaccaccgcacgctgcacgaggtgccgtggctgtacgcgagtgtccacaagccgcaccacaagttcacggcgcccttcgcgtggacgtcccacgcggtgcacccggcggaaatggcgctgcaggccgcgggcgcgatggcggggcctctgctctgggcgcggctgtacggcctgccggtgcgcgcctggtggtgctggctcgccctcgtgcaggcgcagggtgtgatggaccactccgggtatgacctgcccgcgccgctcgactgctttgggatgctgcctgggtttggaggcacgcgcttccacgacgaccaccaccggtactttaccggaaactacgcagccgccctctccctcatcgacgacctgatgggcacccggctgcgagtcgagccccgaggcggtgccggcgctgggcgaggcgcctccgcggtggtcagggccgtggagccggcccggtgggactggaagcgcgacgacttctag

SEQ ID NO: 64 - DNA, 22 bp, Artificial sequence
  Single strand DNA oligonucleotide
atgtggagctattctgtcctcc

SEQ ID NO: 65 - DNA, 20 bp, Artificial sequence
  Single strand DNA oligonucleotide
ccacgcaaagttgagcacga

SEQ ID NO: 66 - DNA, 2001 bp, Emiliania huxleyi
ccgacaccaaaagaattttacaagagatgtgtcgcaagttggtctgactgactgtctcctaacaaacctgccatgcccaaacccaccgcaacctagaccgcaacgaccaacgtcacaaaacaatctctctcccaacgtccaacgagtgggagagagagagagagagagagagagagagagagagagagagagagagagagagagagacgagtggtacgacgaacgcacgaacccatcaatcaaatcaaagttctctcgacatgcacatgcacaacatgtgcatgtgcatgtgcatgtgcatgttgtgcatgtgcatgttgtgtatgtaggaaggtgaagaacagtcaggctcggactggacaccagcgctacctgctaggtaggtaggtgttttggtggacgcaagacaggagatgacggacggcacgctcgctggagcgacacacagacagctcacatggaggacctcatggctggcagtatcatacaaccgaagtacgaccggccctcggagggcgccagctgctctcgactcgaacggctcgcttttgttgaacctcgtgctcctatctaggcgacgtgccaacttccaaggaagtcgcattcgccgagcgttttcgcagcgccgccgcgccccacaaacaaccatgagcggaatcaagcactggctgtccacctggcgcgcgtacgacatcgccaacacgctcgcggccgcctttgtcggtgccagcctctacgcgcggcagcaggagcagcagctctccagccacgccgacccgttcatcgacgcgaccaatgtgacggcgaagccaatgcagcaggcaatggctgaggcgtgggacgccgacccgggcgagatggcggcggccacgtttgagttctggctctttgtgtttgcgatgggcagctcgcccatcgcgtggatcgtcacctacgccttccagtccgtcacgccggcgacgtgggccgcgttcctcccttgggttgcagtcacctttggctcgctcttcaaggggctctccgtcatcgcgtgcgcggcgttcgccgggacctactggtttcacggcctgctgatgctgccgctcgacctgtacgccacgcccgatgcgctcaagcagctgatggtgtccaaggtacagccgaacaagcgcgtcgacccgcacaagcttcccaaggcggtgctcaacatggtgggcaatcttgtctttgtcgccttcccgatcgtcaccgggctcgtgtacgcctcgctcaagacggatcacttcctgcgcgtcgacgagggcctcccgtcgcacaaggagcgctgcctctgcctcgcgtcggtcatcatcggcaacgagatcctcttcttctactcgcactgggcgctgcaccacaaggcgctgtacgccaagatccacaagaagcaccacgagttcaccgcaccgatcgcgctcgtcgccatctactgccacccgatcgagtttgtcctctcggacatcgtgccgctcggcgccgggctgatcgtcgcgcacgcgcacgtcttcttcgccctcatgtggatcgtcaccgccgtcatcggcacgcaggtgcaccactcgggctttcgactgccgtggcactttggtccggatgagcagcctgacttccacgacttccaccaccagaagtttacctgcaactatggccacctcggcatcctcgacgcgctgcacggcacctcgaagatgtacctggaccactgcgccaagctcgccgctgccaagcagaagggcgagtgaggggatttggccgggtttgtctacgtacctctcccggtcagcctggtacagtattccgagccgaagtgcgcttccgctcccttctcccgctggtctctctttgttagtcgtacgtatctctcagcgtctcatccgtcgaactcgcgctgtctgcgagcggcccgaccccgtaccgttaattcttgtgcgcaagccagtgacgccggtcagtatgttttgcgctgcagatacttgttttcaa

SEQ ID NO: 67 - DNA, 20 bp, Artificial sequence
  Single strand DNA oligonucleotide
actttggtccggatgagcag

SEQ ID NO: 68 - DNA, 20 bp, Artificial sequence
  Single strand DNA oligonucleotide
gaccgggagaggtacgtaga

(19) United States
(12) Patent Application Publication
(10) Pub. No.: US 2024/0083843 A1
(43) Pub. Date: Mar. 14, 2024

(54) METHODS OF PRODUCING VITAMIN D

(71) Applicant: YEDA RESEARCH AND DEVELOPMENT CO. LTD., Rehovot (IL)

(72) Inventors: Einat SEGEV, Rehovot (IL); Or ELIASON, Rehovot (IL)

(21) Appl. No.: 18/272,278

(22) PCT Filed: Jan. 14, 2021
(86) PCT No.: PCT/IL2021/050047

§ 371 Date: Jul. 13, 2023

(51) Int. Cl. C07C 401/00; C12N 1/12; C12N 13/00

(52) U.S. Cl. CPC C07C 401/00; C12N 1/12; C12N 13/00

(57) ABSTRACT

A method of producing vitamin D is provided. The method comprising:
(a) growing E. huxleyi under controlled UV-B radiation; and (b) dehydrating the E. huxleyi.

### SEQUENCE LISTING STATEMENT

The ASCII file, entitled 85816 Sequence Listing.txt, created on 13 Jan. 2021, comprising 61,098 bytes, submitted concurrently with the filing of this application is incorporated herein by reference.

### BACKGROUND OF THE INVENTION

The present invention, in some embodiments thereof, relates to methods of producing vitamin D.

Vitamin D is a group of fat-soluble secosteroids responsible for increasing intestinal absorption of calcium, magnesium, and phosphate, and many other biological effects. In humans, the most important compounds in this group are vitamin D3 (also known as cholecalciferol) and vitamin D2 (ergocalciferol).^{1,2} In mammals, vitamin D regulates the absorption of calcium in the gut necessary for bone formation and replenishment⁹.

To date, vitamin D is consumed as a recommended food supplement worldwide as 20-80% of US, Canadian and European women and men are vitamin D deficient^{3,4}. Deficiency results in impaired bone mineralization and bone damage which leads to bone-softening diseases, including rickets in children and osteomalacia in adults. The biosynthesis of vitamin D requires Ultra Violet B (UV-B) radiation achieved by exposure to sunlight¹⁰.

Although vitamin D is present naturally in only a few foods, it is commonly added as a fortification in manufactured foods. In some countries, staple foods are artificially fortified with vitamin D.

In general, vitamin D3 is found in animal source foods, particularly fish, meat, offal, egg and dairy.^{[135]} Vitamin D2 is found in fungi and is produced by ultraviolet irradiation of ergosterol.^{[136]} The vitamin D2 content in mushrooms and Cladina arbuscula, a lichen, increase with exposure to ultraviolet light and is emulated by industrial ultraviolet lamps for fortification [Wang T, Bengtsson G, Kärnefelt I, Björn LO (September 2001). “Provitamins and vitamins D₂and D₃ in Cladina spp. over a latitudinal gradient: possible correlation with UV levels”. Journal of Photochemistry and Photobiology. B, Biology (Submitted manuscript). 62 (1-2): 118-22; Haytowitz Vitamin D in Mushrooms: www(dot)ars(dot)usda(dot)gov/ARSUserFiles/80400525/Articles/AICR09_Mushroom_VitD.pdf].

Coccolithophores are microalgae, which constitute one of the major groups that produce Ca-rich minerals (a process termed “calcification), similar to an external shell. Coccolithophores cover their cell with coccoliths, which are microscopic platelets made of a CaCO₃ mineral called calcite (FIG. 1), and are considered to be the most productive calcifiers globally^{11,16,21}. The platelets are produced intracellularly and ejected onto the cell surface. With the death of the cell, the coccoliths sink to the bottom of the ocean and form carbonate sediments. This transport of CaCO₃ to the deep ocean is an important process of carbon sequestration²².

Among the numerous species of coccolithophores inhabiting the world oceans, Emiliania huxleyi is the most ubiquitous one. E. huxleyi is found almost everywhere in modern oceans, playing key roles in the global oxygen, carbon and sulfur cycles^{11,16}.

E. huxleyi is known to grow in strong light environments, and is often found in high concentrations close to the water surface, sometimes at shallow depths of only 10 to 30 meters¹⁸. Of note, although water are an efficient ultraviolet radiation filter, significant amounts of UV-B radiation can penetrate depths in which E. huxleyi thrives¹⁹. In an experiment conducted in the early 1980's, E. huxleyi was noted to produce the vitamin D precursor ergosterol, and to contain measurable amounts of previtamin D2 after exposure to artificial sunlight²⁰.

Additional background art includes:

### U.S. Patent Publication No. 20180078521

Japlet and Jakobsen Front. Plant Sci. 2013 May 13; 4:13 doi: 10.3389/fpls.2013.00136. eCollection 2013.
Guan and Gao 2010 Environmental and Experimental Botany 67(3):502-508.

### SUMMARY OF THE INVENTION

According to an aspect of some embodiments of the present invention there is provided a method of producing vitamin D, the method comprising:
(a) growing E. huxleyi under controlled UV-B radiation; and
(b) dehydrating the E. huxleyi;

According to an aspect of some embodiments of the present invention there is provided a method of monitoring vitamin D production in E. huxleyi, the method comprising:
(a) growing the E. huxleyi under controlled UV-B radiation;
(b) dehydrating the E. huxleyi; and
(c) determining a level of expression of a gene in a vitamin D biosynthetic pathway of the E. huxleyi prior to and/or following step (b), the level being indicative of vitamin D production.

According to an aspect of some embodiments of the present invention there is provided a method of culturing E. huxleyi, the method comprising supplementing an E. huxleyi culture with bacteria of the species Phaeobacter inhibens and growing the culture under controlled UV-B radiation.

According to some embodiments of the invention, the vitamin D comprises vitamin D2 and vitamin D3.

According to some embodiments of the invention, values of the vitamin D are about the same prior to and following dehydration.

According to some embodiments of the invention, the dehydrating is effected under conditions which preserve the level of vitamin D.

According to some embodiments of the invention, the growing is effected in a closed setting.

According to some embodiments of the invention, the growing is effected in the presence of a defined bacterial environment.

According to some embodiments of the invention, the defined bacterial environment comprises no more than 50 bacterial species.

According to some embodiments of the invention, the bacterial species comprises or consists of Phaeobacter inhibens.

According to some embodiments of the invention, the method does not comprise an extraction step.

According to some embodiments of the invention, the bacteria is separated from the E. huxleyi by a barrier allowing fluid and solute communication between the E. huxleyi and the bacteria.

According to some embodiments of the invention, the growing is effected at a pH above 7.

According to an aspect of some embodiments of the present invention there is provided a dry biomass of E. huxleyi.

According to some embodiments of the invention, the dry biomass comprises defined bacteria.

According to some embodiments of the invention, the dry biomass comprises no more than 50 bacterial species.

According to some embodiments of the invention, the dry biomass comprises Phaeobacter inhibens.

According to some embodiments of the invention, the dry biomass is axenic.

According to some embodiments of the invention, the dry biomass is free of bacteria.

According to an aspect of some embodiments of the present invention there is provided a two-phase system for growing E. huxleyi, the system comprising an E. huxleyi culture and bacteria which support the growth of the E. huxleyi, the bacteria and E. huxleyi being separated by a barrier allowing fluid and solute communication between the E. huxleyi and the bacteria.

According to some embodiments of the invention, the bacteria comprise defined bacteria.

According to some embodiments of the invention, the two-phase system comprises no more than 50 bacterial species.

According to some embodiments of the invention, the bacteria comprises or consists of Phaeobacter inhibens.

According to an aspect of some embodiments of the present invention there is provided a method of improving nutrition, the method comprising administering to a subject in need thereof an effective amount of the dry biomass as described herein, thereby improving the nutrition of the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a Scanning electron microscopy (SEM) image of E. huxleyi (CCMP3266) pure algal culture. The calcium carbonate coccoliths can be seen surrounding the cells.

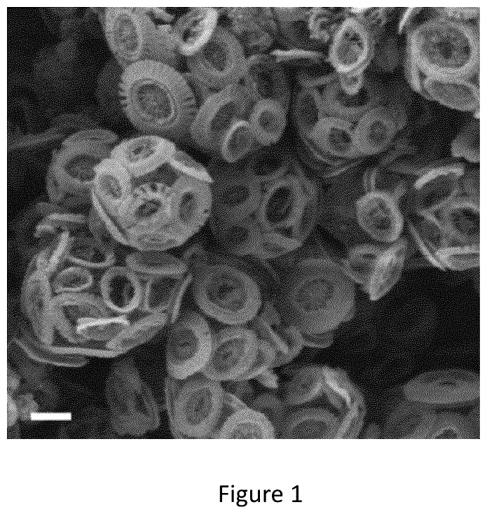

FIG. 2 shows E. huxleyi genes encoding the canonical pathways of sterol synthesis in land plants, fungi, and vertebrates. Following a bioinformatic search, genes encoding for enzymes were found in E. huxleyi. Some genes were not found (ERG27 and LAS). Enzymes surrounded with a bold black line are putatively encoded by bacterial genes in P. inhibens.

FIG. 3 shows that E. huxleyi exposure to controlled UV-B results in detectable levels of vitamin D2 and its precursor ergosterol. Short exposure corresponds to 1 hour, and long to 5 hours. The results are not normalized to cell numbers, and therefore cannot be compared. As a positive control, cultures were cultivated under natural sunlight. As a negative control, cultures were grown in a standard growth chamber lacking UV. These results are comparable since the biomass used for extraction was similar in all experiments.

FIG. 4 shows the relative quantification of key genes in the sterol and vitamin D synthesis pathway in E. huxleyi, comparing UV and regular light regimes. The location of each gene along the sterol biosynthesis pathway can be seen in FIG. 2.

FIG. 5 shows a view of the UV-B experimental setup. A UV-B fluorescent lamp (Exo-Terra REPTILE UVB150) was installed in the chamber along with an intensity reducing filter. UV-B emittance was confirmed using an Ocean Optics HR4000 spectrometer. The open flask contains plastic beads which develop coloration when exposed to UV, thus acting as an extra validation for UV penetration of the glass.

FIG. 6 shows vitamin D3 detection after exposure to UV radiation by UPC²; The Figure shows UPC² analysis of E. huxleyi vitamin D extracts. Upper panel showing results for vitamin D3 standard. Lower panel showing extract of an algal sample with peaks matching in retention time and mass to the vitamin D3 standard. This data demonstrates for the first time the identification of vitamin D3 in the microalga E. huxleyi.

### DESCRIPTION OF SPECIFIC EMBODIMENTS OF THE INVENTION

The present invention, in some embodiments thereof, relates to methods of producing vitamin D.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Vitamin D is an essential vitamin for the health and growth of bones. It is also important for calcium and phosphorus metabolism. Vitamin D deficiency can result in skeletal diseases, such as rickets and osteomalacia among other medical conditions which are listed hereinbelow. Lately, it has also been reported that vitamin D can prevent infection and ameliorate symptoms associated with coronavirus infection as in the case of COVID19.

There are not many natural sources for vitamin D, therefore the amount of time in sunlight is the important source of vitamin D3, however this is often insufficient.

Whilst conceiving embodiments of the invention, and whilst looking for alternative sources for vitamin D, the present inventors were able to construct the sterol synthesis pathway in the microalga, E. huxleyi. Surprisingly, this pathway was found to lead to vitamin D3 production, a vitamin that is known to be synthesized primarily in animals and is not common in plants nor algae. Importantly, three different canonical pathways for sterol synthesis are found in mammals, plants and fungi. In the newly discovered sterol pathway in E. huxleyi all three pathways have been identified.

Under controlled UV-B radiation, the present inventors were able to induce production of vitamins D2 and D3 as well as other sterols. In the presence of bacteria, the growth of the alga was induced and vitamins yields increased.

This is the first evidence for the production of a nutritional dry biomass of E. huxleyi which provides multiple benefits, i.e., vitamin D2, vitamin D3, calcium (by virtue of its coverage with coccoliths) and other sterols, rendering it an important supplement for improving human and animal nutrition.

Thus, according to an aspect of the invention there is provided a method of producing vitamin D, the method comprising:
(a) growing E. huxleyi under controlled UV-B radiation; and
(b) dehydrating the E. huxleyi;

As used herein “vitamin D” refers to a steroid hormone; the major compounds in this group are vitamin D3 (cholecalciferol) and vitamin D2 (ergocalciferol).

According to a specific embodiment, vitamin D is vitamin D3 and optionally vitamin D2, typically both.

As used herein “producing” refers to a crude production in the alga which is terminated by separating and drying the biomass without further extraction. However, purified extracts may also be envisaged. “Producing”, typically refers to a large scale production (e.g., at least 40 gr from 1000 liters culture or in a small scale at least 1.4 grams from 40 liters of culture) for commercial purposes.

As used herein “E. huxleyi” refers to Emiliania huxleyi (or as often referred to herein as “the alga”), a species of coccolithophore found in almost all ocean ecosystems from the equator to sub-polar regions, and from nutrient rich upwelling zones to nutrient poor oligotrophic waters. Like other coccolithophores, E. huxleyi is a single-celled phytoplankton covered with uniquely ornamented calcite disks called coccoliths.

According to a specific embodiment, the E. huxleyi is of a cultivated variety for improved and reproducible performance, such as described in the Examples section which follows, available from Bigelow National Center for Marine Algae and Microbiota.

According to a specific embodiment, the alga is non-transgenic.

According to another specific embodiment, the alga is transgenic.

Methods of transforming E. huxleyi can be found in U.S. Pat. No. 10,017,796, which is hereby incorporated in its entirety.

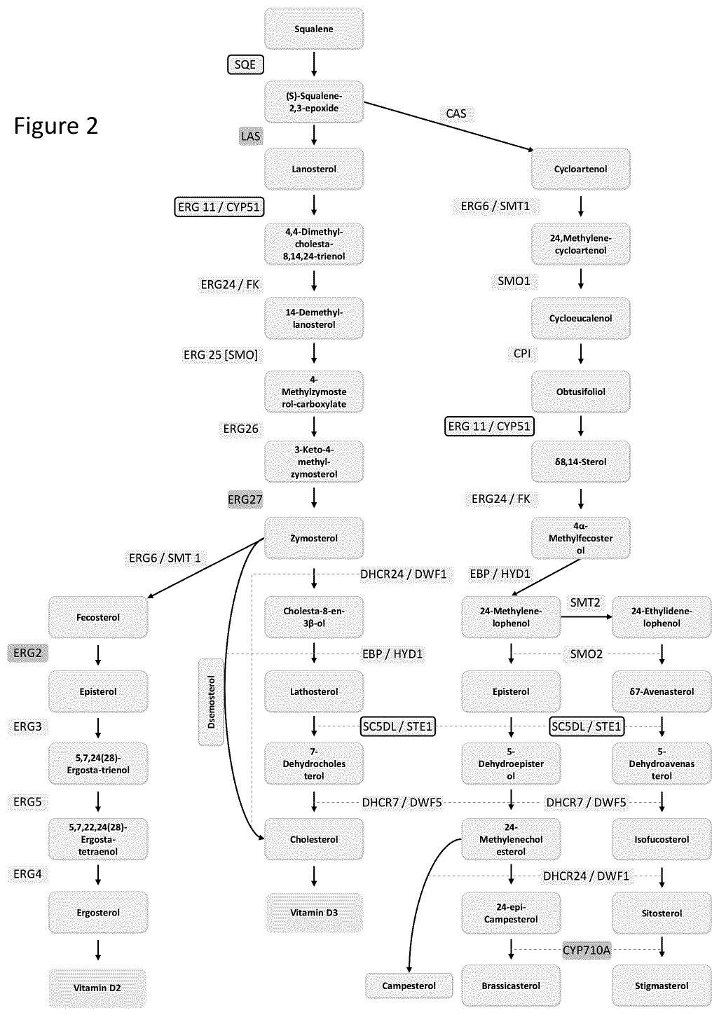

The alga can be transformed with a gene which elevates the sterol pathway as depicted in any one of the genes of FIG. 2 and or any gene which elevates other nutritional values such as Omega-3 production, as supported by U.S. Pat. No. 10,017,796 (supra). Alternatively or additionally, the microalga can be transformed for elevated expression of a gene which facilitates culturing to provide higher yield, vigor, survival under stress conditions or the like.

In one embodiment, the alga expresses one or more heterologous nucleic acid sequences which encode expression product(s).

As used herein, the words “nucleic acid”, “nucleic acid sequence”, “nucleotide”, or “polynucleotide” are intended to include DNA molecules (e.g. cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in genomic sequence, or may comprise only a coding sequence as in cDNAs, and/or may include cDNAs in combination with regulatory sequences. In one embodiment of the various aspects of the invention, cDNA sequences synthetic (deduced) open reading frames, analogous to cDNA are preferred.

As used herein, “transgenic”, “transgene” or “recombinant” refers, for example, to a nucleic acid sequence, an expression cassette, gene construct, a vector or an autonomous replicating element such as an artificial chromosome comprising the nucleic acid sequence or an organism transformed with the nucleic acid sequences, expression cassettes or vectors according to the invention, all those constructions brought about by recombinant methods in which either (a) the nucleic acid sequences encoding proteins useful in the methods of the invention, or (b) genetic control sequence(s) which is operably linked with the nucleic acid sequence according to the invention, for example a promoter, or (c) a) and b) are not located in their natural genetic environment or have been modified by recombinant methods, such as mutagenesis, it being possible for the modification to take the form of, for example, a substitution, addition, deletion, inversion or insertion of one or more nucleotide residues. The natural genetic environment is understood as meaning the natural genomic or chromosomal locus in the original microalgae or the presence in a genomic library.

A transgenic alga for the purposes of the invention is thus understood as meaning an alga which comprises within its nuclear and or plastidial genome a heterologous polynucleotide. The heterologous polynucleotide is may be stably integrated within the genome such that the polynucleotide is passed on to successive generations. The heterologous polynucleotide may be integrated into the genome alone or as part of a recombinant DNA construct.

It will be appreciated that the alga can be alternatively or additionally modified also by other means such as by chemical or genetic manipulation (e.g. drug treatment, gene knockdown/knockout, genome editing) such as to achieve modified lipid content (not necessarily sterol), alkenone content, carotenoid content, fucoxanthin content, photosynthetic rate, growth rate, gene expression, protein expression, and the like. as a means to create a superior variety.

Inocula of the alga can be commercially purchased from Bigelow National Center for Marine Algae and Microbiota (www(dot)ncma(dot)bigelow(dot)org/).

As mentioned, the method contemplates growing or cultivating the E. huxleyi under controlled UV-B radiation.

As used herein “controlled UV-B radiation” means artificial radiation which is in the range of 280-315 nm. For example this can be achieved by a UV-B fluorescent lamp (Exo-Terra REPTILE UVB150). For larger settings it is possible to use “Philips TL20W/01RS narrowband UV-B tubes” or their broadband version. In addition, a number of growth chambers such as the Percival growth chamber, can be equipped with a plurality of Exo-Terra lights.

Alternatively, the alga is subjected to sunlight which is supplemented by artificial UV-B radiation.

According to a specific embodiment, cultivation is done in a closed setting which is not subjected to sunlight (also referred to herein as indoors).

According to a specific embodiment culturing in closed setting is done in fermenters.

According to a specific embodiment, the cultivation is done in open settings subjected to sunlight in which case UV-B radiation is supplemented.

According to a specific embodiment, cultivation is done outdoors.

The medium is typically comprised of seawater or artificial seawater. According to a specific embodiment nutrients are added such as a mix called L1 (www(dot)doi(dot)org/10.2216/i0031-8884-32-3-234.1) in which silica is omitted, resulting in L1-Si which is commonly used for growing calcifying algae.

Most commercial production techniques use large open ponds, taking advantage of natural sunlight, which is free. These systems have a relatively low surface area to volume ratio with corresponding low cell densities. Hence in such systems impellers or stirrers are typically used to benefit the entire photosynthetic depth of the water column. The need to exclude contaminating organisms in open ponds, typically restricts the usefulness of open ponds to a limited number of algae that thrive in conditions not suitable for the growth of most organisms. For example, Dunaliella salina can be grown at very high salinities. Apt K E et al, “Commercial Developments in Microalgal Biotechnology,” J Phycol. 35:215-226 (1999). Of course measures are taken to comply the salinity conditions for other species used in cultivation. However, in the case of endophytes or epiphytes, this should not pose a problem as their natural environment is that of the alga.

Enclosed photobioreactors, such as tubular photobioreactors and raceway tanks, are an alternative outdoor closed culture technology that utilize transparent tubes enclosing the culture minimizing contamination. They provide a very high surface to volume ratio, so cell densities are often much higher than those that can be achieved in a pond.

Numerous designs have also been constructed for the indoor, closed culture of algae using electric lights for illumination. Ratchford and Fallowfield (1992) “Performance of a flat plate, air lift reactor for the growth of high biomass algal cultures,” J Appl. Phycol. 4: 1-9; Wohlgeschaffen, G D et al. (1992) “Vat incubator with immersion core illumination—a new, inexpensive set up for mass phytoplankton culture,” J Appl. Phycol. 4:25-9; Iqbal, M et al. (1993) “A flat sided photobioreactor for culturing microalgae,” Aquacult. Eng. 12:183-90; Lee and Palsson (1994) “High-density algal photobioreactors; using light-emitting diodes,” Biotechnol. Bioeng. 44:1161-7.

In some embodiments, the culture is supplemented with bacteria which promote algal growth.

Thus, according to an aspect of the invention, there is provided a method of culturing E. huxleyi, the method comprising supplementing an E. huxleyi culture with bacteria of the species Phaeobacter inhibens and growing the culture (co-culture) under controlled UV-B radiation.

The culture can be first “cleaned” from bacteria which are endogenous to the culture. In order to make algal cultures axenic (i.e. without bacteria) a mixture of antibiotics is used to treat the culture during several passages. According to some embodiments, the antibiotics used are penicillin (e.g., 0.1 mg/ml) and/or streptomycin (e.g., 0.05 mg/ml), during a number of passages (e.g., 2-4). Bacterial presence at the end of the treatment is tested using microscopy, flow cytometry and plating. If necessary further passages include the addition of different antibiotic or antibiotics mix e.g., gentamycin (e.g., 50 μg/ml), kanamycin (e.g., 350 μg/ml) and/or ampicillin (e.g., 300 μg/ml). After successful removal of bacteria, algae are grown in antibiotic-free medium, and bacterial absence is verified periodically.

According to a specific embodiment, the bacteria comprises Phaeobacter inhibens, e.g., DSM17395.

According to a specific embodiment, the initial algal concentration is 300 cells/ml, and bacterial concentration of 10 CFU/ml. Bacterial cells are measured as CFU (colony forming units), a common unit for bacterial measurements. According to other embodiments the following ranges are contemplated 10-100 CFU/ml and respective 300-3000 algal cells/ml. However, any range is contemplated provided the culture time.

Methods of growing Phaeobacter inhibens are well known in the art. For example, for obtaining an inoculoum bacteria, frozen bacterial stocks are plated on ½ YTSS agar plates containing yeast extract, tryptone, sea salts and agar in distilled water. A single colony is transferred into a liquid medium containing sea water enriched with glucose, Na₂SO₄ (Merck), NH₄Cl and KH₂PO₄. Liquid bacterial cultures are cultivated at 30° C. for 24 hours, shaking at 130 rpm, from which the algal cultures are inoculated.

According to some embodiments the pH of the culture is above 7 to prevent calcium dissolution, e.g., 7.2-9, 7.5-9, 7.8-9, 8-9, 8-8.5, e.g., 8.2.

According to a specific embodiment, growing is effected in the presence of a defined bacterial environment.

As used herein “defined bacterial environment” refers to a culture in which the majority of the bacteria are known and are intended to be present in the culture. According to a specific embodiment, the culture is devoid of pathogenic bacteria (causing diseases in human beings for instance).

According to a specific embodiment, the defined bacterial environment comprises no more than (100, 50 or 20) bacterial species.

According to a specific embodiment, the bacterial species comprises or consists of Phaeobacter inhibens.

According to a specific embodiment, the cultivation is done under sterile conditions.

According to a specific embodiment, the cultivation is done under non-sterile conditions.

According to a specific embodiment, the culture is free of bacteria.

According to a specific embodiment, the bacteria is separated from the algae by a barrier allowing fluid and solute communication between said E. huxleyi and said bacteria. Hence, culturing can be done in a two-phase system where the algae are separated from the exogenously added bacteria by a barrier typically for simpler harvesting of the algae without the bacteria when cultivation terminates.

Thus according to an aspect of the invention, there is provided a two-phase system for growing E. huxleyi, the system comprising an E. huxleyi culture and bacteria which support the growth of said E. huxleyi, said bacteria and E. huxleyi being separated by a barrier allowing fluid and solute communication between said E. huxleyi and said bacteria.

One way to achieve a two-phase system is by encapsulating the alga separately from the bacteria (or the other way around). Of course measures should be taken to employ a barrier which still allows solute passage as well as UV-B penetration.

Encapsulated microalgae have been described for several purposes such as feed, cosmetics, as well as oxygen producers for co-cultured heterotrophic cells (Bloch K, Papismedov E, Yavriyants K, Vorobeychik M, Beer S, Vardi P. Photosynthetic oxygen generator for bioartificial pancreas. Tissue Eng. 2006 Feb; 12(2):337-44. Kitcha S, Cheirsilp B. Enhanced lipid production by co-cultivation and co-encapsulation of oleaginous yeast Trichosporonoides spathulata with microalgae in alginate gel beads. Appl Biochem Biotechnol. 2014 May; 173(2):522-34. doi:10.1007/s12010-014-0859-5. de-Bashan L E, Bashan Y. Joint immobilization of plant growth-promoting bacteria and green microalgae in alginate beads as an experimental model for studying plant-bacterium interactions. Appl Environ Microbiol. 2008 Nov; 74(21):6797-802. doi: 10.1128/AEM.00518-08).

Encapsulated microalgae have been shown to preserve microalgae growth rate and even accelerate cell proliferation and microalgae compounds production (Joo D S, Cho M G, Lee J S, Park J H, Kwak J K, Han Y H, Bucholz R. New strategy for the cultivation of microalgae using microencapsulation. J Microencapsul. 2001 Sep-Oct; 18(5):567-76. de-Bashan L E, Bashan Y. Immobilized microalgae for removing pollutants: review of practical aspects. Bioresour Technol. 2010 Mar; 101(6):1611-27. doi: 10.1016/j.biortech.2009.09.043). Such a possibility is of crucial advantage in the food industry as well as in any other algae based industrial systems.

It will be appreciated that a two-phase system may be still characterized by ano other parameters as described herein, such as defined bacteria, level of sterility and so forth.

Regardless of the presence or absence of bacteria, after a predetermined time in culture, the alga is isolated, collected and dried, and is typically kept in the dark at room temperature.

Culturing lasts between 12-18 days. Culturing time depends on culture volume and initial cell concentration in the culture.

Separation of the biomass can be done using various methods known in the art. These include, but are not limited to, flocculation followed by sedimentation, centrifugation and filtration.

As used herein “dehydration” refers to water removal aiming to reduce moisture content and water activity and consequently stabilization of the product by inhibiting microbial growth and enzymatic activity and slowing chemical reactions.

Ultimately, the process ends with algal dry biomass (e.g., powder) in which the vitamin D as well as other nutritional values are preserved, i.e., about 90% or more of the vitamin D levels (D3 and optionally D2) are about the same as prior to dehydration.

The dehydration method depends on the consequent use. For instance, when used for human or animal use the following methods can be employed: Freeze-drying (FD), hot-air drying (HAD), heat pump drying, convective drying (CD), connective drying in thin layer, infrared drying (ID). Spray drying (SD), vacuum drying (laboratory scale), spouted bed dryer (SBD), conventional tray drying (CTD), perpendicular airflow drying, discontinuous tray drying, tray drying, tray drying with air circulation, fluid-bed drying with alginate cells and atomization drying.

According to some embodiments, the algal sterol content and calcium carbonate coverage on the cell wall is undisrupted following dehydration.

According to a specific embodiment, the process does not comprise an extraction step, in which the vitamin D is separated from the biomass.

However, in other embodiments, the processing may include a further step of extraction of the vitamin D. According to such embodiment, the algal material may be homogenized in the presence of liquid nitrogen and analytes are extracted at 75° C. for 60 min with chloroform/methanol such as at the ratios specified in the Examples section which follows.

The percentage of water following drying is not more than 1-5%, e.g., 3%.

Monitoring vitamin D production can be done using methods which are well known in the art (such as by using reverse phase lipid chromatography, e.g., Acquity® UPC2TM system) and/or optionally assisted by markers which essentially compose the biosynthetic pathway of FIG. 2 and suggested primers (non-limiting) are provided as well.

Thus, according to an aspect of the invention there is provided a method of monitoring vitamin D production in E. huxleyi, the method comprising:
(a) growing the E. huxleyi under controlled UV-B radiation;
(b) dehydrating the E. huxleyi; and
(c) determining a level of expression of a gene in a vitamin D biosynthetic pathway of said E. huxleyi prior to and/or following step (b), the level being indicative of vitamin D production.

Determining the gene expression can be done in the absence of drying at all.

Thus for example:
Squalene moonxygenase. E.C: 1.14.14.17 (SEQ ID NO: 1) and suggested primers (SEQ ID NO: 2-3);
DWF1. Delta24-sterol reductase [EC:1.3.1.72 1.3.1.-] (SEQ ID NO: 4) and suggested primers (SEQ ID NO: 5-6);
ERG3. Delta7-sterol 5-desaturase [EC:1.14.19.20], three different genes SEQ ID NO: 12): and suggested primers (SEQ ID NO: 13-14);
DWFS. 7-dehydrocholesterol reductase [EC:1.3.1.21] (SEQ ID NO: 15) and suggested primers (SEQ ID NO: 16-17);
Delta14-sterol reductase [EC:1.3.1.70] (also called FK) (SEQ ID NO: 18) and suggested primers: (SEQ ID NO: 19-20);
Cycloartenol/lanosterol synthase [EC:5.4.99.8/5.4.99.7] (SEQ ID NO: 21) and suggested primers: (SEQ ID NO: 22-23);
ERG6. SMT1. sterol 24-C-methyltransferase [EC:2.1.1.41] (SEQ ID NO: 24) and suggested primers: (SEQ ID NO: 25-26);
Cycloeucalenol cycloisomerase [EC:5.5.1.9] two genes: (SEQ ID NO: 27-28) and suggested primers: (SEQ ID NO: 29-30);
STE1. Delta7-sterol 5-desaturase [EC:1.14.19.20] three genes (SEQ ID NO: 31-33) and suggested primers: (SEQ ID NO: 34-35);
CYP51G1 [EC:1.14.13.70] (SEQ ID NO: 36) and suggested primers: (SEQ ID NO: 37-38);
1.1.1.170 (SEQ ID NO: 39) and suggested primers: (SEQ ID NO: 40-41);
ERG2/EBP/HYD1 (E.C: 5.3.3.5) (SEQ ID NO: 42) and suggested primers: (SEQ ID NO: 43-44);
ERGS (SEQ ID NO: 45) and suggested primers: (SEQ ID NO: 46-47);
ERG 4 E.C: 1.3.1.71 (SEQ ID NO: 48) and suggested primers: (SEQ ID NO: 49-50);
farnesyl-diphosphate farnesyltransferase EC:2.5.1.21 (SEQ ID NO: 51) and suggested primers: (SEQ ID NO: 52-53);
Putative Lipase, JGI ID 438299 (SEQ ID NO: 54) and suggested primers: (SEQ ID NO: 55-56);
SMO4 (SEQ ID NO: 57, 60, 63, 66) and suggested primers: (SEQ ID NO: 58-59, 61-62, 64-65 and 67-68);

Methods useful for monitoring the expression level of specific genes are well known in the art and include RT-PCR, semi-quantitative RT-PCR, Northern blot, RNA in situ hybridization, Western blot analysis and immunohistochemistry.

An increase in expression being indicative of induction of the pathway.

As used herein “increase” refers to at least 10%, 20%, 30%, 40%, 50%, 60%, 70%, 80%, 90% or more, say 1.5 fold, 2 fold, 5 fold, 10 fold. 50 fold or more compared to the same level of expression (or vitamin D) in a control biomass grown under the same conditions without controlled UV-B radiation.

Thus, the present teachings provide for a dry biomass obtainable according to the method described herein.

This biomass or formulation comprising same which can be in the form of a powder, paste or emulsion can be used in various cosmetic and clinical applications.

Thus, according to an aspect of the invention there is provided a method of improving nutrition, the method comprising administering to a subject in need thereof an effective amount of the dry biomass of any one of claims 13-18, thereby improving the nutrition of the subject.

As used herein “a subject in need thereof” can be a human or animal who suffers from vitamin D deficiency or is at risk of developing vitamin D deficiency or can benefit from supplementation in vitamin D [e.g., as in the case of Coronavirus (e.g., SARS-CoV-2) infection].

“Vitamin D deficiency” is generally defined as a condition in a human patient or other mammal in which serum 25-hydroxyvitamin D levels is below 30 ng/mL (see National Kidney Foundation guidelines, NKF, Am. J. Kidney Dis. 42:S1-S202 (2003), incorporated herein by reference). “Vitamin D deficiency” includes “vitamin D insufficiency,” defined as serum 25-hydroxyvitamin D of at least 16 ng/mL and less than 30 ng/mL, “mild” vitamin D deficiency, defined as serum 25-hydroxyvitamin D of 5-15 ng/mL, and “severe” vitamin D deficiency, defined as serum 25-hydroxyvitamin D below 5 ng/mL.

As used herein, the term “vitamin D replete” is defined as a condition in a human patient or other mammal in which serum 25-hydroxyvitamin D levels is at or above 30 ng/mL.

The term “at risk” as used herein generally refers to those patient populations having characteristics or diseases associated with vitamin D deficiency. Specific examples include, but are not limited to, subjects with Stage 1, 2, 3, 4 or 5 chronic kidney disease; infants, children and adults that do not drink vitamin D fortified milk (e.g. lactose intolerant subjects, subjects with milk allergy, vegetarians who do not consume milk, and breast fed infants); subjects with rickets; subjects with dark skin (e.g., in the U.S., 42% of African American women between 15 and 49 years of age were vitamin D deficient compared to 4% of white women); the elderly (who have a reduced ability to synthesize vitamin D and also are more likely to stay indoors); chronically or acutely and severely ill adults (who are likely to stay indoors, in hospitals, in intensive care facilities, institutional and assisted-care facilities including subjects with Alzheimer's disease or mentally ill); subjects who cover all exposed skin (such as members of certain religions or cultures); subjects who always use sunscreen (e.g., the application of sunscreen with a Sun Protection Factor (SPF) value of 8 reduces production of vitamin D by 95%, and higher SPF values may further reduce vitamin D); subjects with fat malabsorption syndromes (including but not limited to cystic fibrosis, cholestatic liver disease, other liver disease, gallbladder disease, pancreatic enzyme deficiency, Crohn's disease, inflammatory bowel disease, sprue or celiac disease, or surgical removal of part or all of the stomach and/or intestines); subjects with inflammatory bowel disease; subjects with Crohn's disease; subjects who have had small bowel resections; subjects with gum disease; subjects taking medications that increase the catabolism of vitamin D, including phenytoin, fosphenytoin, phenobarbital, carbamazepine, and rifampin; subjects taking medications that reduce absorption of vitamin D, including cholestyramine, colestipol, orlistat, mineral oil, and fat substitutes; subjects taking medications that inhibit activation of vitamin D, including ketoconazole; subjects taking medications that decrease calcium absorption, including corticosteroids; subjects with obesity, diabetes mellitus, insulin resistance syndrome, endothelial dysfunction (vitamin D deposited in body fat stores is less bioavailable); subjects with osteoporosis; postmenopausal women; individuals with cardiovascular disease, atherosclerosis, and/or heart failure; and/or critically-ill hospitalized subjects, subjects suffering from cancer or hyperthyroidisim including athyroidism, secondary hyperparathyroidism, hyperparathyroidism secondary to chronic kidney disease (Stage 3, 4 or 5) and hyperparathyroidism secondary to vitamin D deficiency.

Vitamin D deficiency is associated with a host of additional diseases and disorders, including secondary hyperparathyroidism, parathyroid gland hyperplasia, hypocalcemia, psoriasis, chronic kidney disease (CKD), and metabolic bone diseases such as fibrogenesis imperfecta ossium, osteitis fibrosa cystica, osteomalacia, rickets, osteoporosis, osteopenia, osteosclerosis, renal osteodystrophy, and extraskeletal calcification. The methods in accordance with the present disclosure also are useful for treating or preventing diseases or disorders associated with vitamin D deficiency.

The dried biomass such as in the form of powder can be combined with ordinary foods to enhance the vitamin D content of the foods. For example, the compositions can be mixed with drinks, food supplements, nutritional supplements, snacks, bars, and virtually any other food, nutritional product or drink that is desired to be supplemented with vitamin D. Thus, the invention specifically includes food substances of specific types combined with the compositions of the invention in specified forms and quantities. The compositions of the invention may be combined with one or more substances to increase the nutritive value of the composition, such substances including, but not limited to, vitamins, minerals, amino acids, proteins, and combinations thereof.

In the cosmetics field, the dry biomass according to the invention can advantageously be used alone or in in combination with retinoids, with corticosteroids, in combination with free-radical scavengers, with .alpha.-hydroxy or .alpha.-keto acids or derivatives thereof, or alternatively with ion-channel blockers, the various products taken in combination with the biomass of the present invention being as defined above.

The present invention is thus also directed towards a cosmetic composition containing, in a cosmetically acceptable support and the biomass as described herein. This cosmetic composition can be in particular in the form of a cream, a milk, a lotion, a gel, microspheres or nanospheres or lipid vesicles or polymer vesicles, a soap or a shampoo.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531;

5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Gene curation—Genes were manually defined according to Feldmesser et al.

www(dot)bmcgenomi cs(dot)bi omedcentral(dot)com/arti cl es/10.1186/1471-2164-15-148 with the following changes: The initial input was with the protein sequences found in tomato, according to Sonawane et al. (www(dot)nature(dot)com/articles/nplants2016205) The pathway of Steroid Biosynthesis (map00100) in KEGG (Kanehisa et al. www(dot)academic(dot)oup(dot)com/nar/article/49/D1/D545/5943834) was followed. If a gene was predicted in E. huxleyi, its sequence was used as the initial input, and its definition was verified. If not, various algae and plants species, yeast, or human were used as input. Additionally, all relevant genes were compared to E. huxleyi' s genome in a local blast, and gene definitions were corrected or performed with transcriptome reads and ESTs.

Growth conditions—E. huxleyi (CCMP3266) cultures (originally from Bigelow National Center for Marine Algae and Microbiota (www(dot)ncma(dot)bigelow(dot)org/) were grown in 50 ml artificial sea water enriched with Ll-Si²⁸, in borosilicate Erlenmeyer flasks. Growth was conducted at 18° C. under light intensity of 130 μm photons m⁻²s⁻¹ with light:dark cycle of 16:8 hours. Cultures were initiated with a final inoculum of 300 cells ml⁻¹. Cultures subjected to constant UV radiation were grown in an identical conditions, and were supplemented with a UV-B fluorescent lamp (Exo-Terra REPTILE UVB150).

Bacterial influence on algal sterol composition was achieved by inoculating the algal cultures with Phaeobacter inhibens (DSM17395) to a final CFU concentration of 50 ml⁻¹. For growing bacteria, frozen bacterial stocks were plated on ½ YTSS agar plates containing 2 gr yeast extract, 1.25 gr tryptone, 20 gr sea salts (Sigma Aldrich) and 16 gr agar (BD biosciences) in 1 L distilled water. Plates were incubated for 24 hours at 30° C. A single colony was transferred into liquid medium containing 10 ml of sea water enriched with 5.5 mM glucose (Sigma Aldrich), 33 mM Na₂SO₄ (Merck), 5 mM NH₄Cl (Sigma Aldrich) and 2 mM KH₂PO₄ (Carl Roth)²⁸. Liquid bacterial cultures were cultivated at 30° C. for 24 hours, shaking at 130 rpm, from which the algal cultures were inoculated.

After 10 days of growth, algal cultures were centrifuged, the pellet collected and lyophilized, and were kept at the dark in room temperature until analysis.

### Vitamin D Extraction and Profiling

### Protocol:

During all steps, protect samples from light as much as possible.
1. For each dry sample add:
   108 μl of 55% KOH in DDW
   192 μl EtOH (containing 50 ng of internal standard)
   60 of 8.9% NaCl, 7.4% ascorbic acid in DDW (e.g. 72 mg NaCl & 60 mg asc acid in 810 μl DDW for 12 samples+1 excess).
2. Homogenize sample, stir (vortex) at room temperature for 18 hours, protected from light.
3. Add 400 μl % NaCl and then 300 μl of 20% Ethyl acetate in heptane.
4. Vortex extensively (30 seconds) and centrifuge 3 minutes.
5. Transfer upper phase to a new vial, repeat two additional times.
6. Evaporate. Samples can be kept in −80° C.
7. Dissolve samples with 200 μl 0.5% IPA in hexane, vortex vigorously and centrifuge for 10 minutes.
8. Condition silica column (Phenomenex Strata SI-silica 55 μm 70 A) by washing it with 1 ml of 50%/50% chloroform and IPA.
9. Wash tube twice with 1 ml hexane. The color of sorbent should change from white to grey.
10. Load sample into tube and allow it enter the sorbent layer.
11. Wash sample by adding 0.5 ml of 0.5% IPA in hexane and discarding the flow through.
12. Add 2.5 ml (1, 1, 0.5 successively) of 2.5% IPA in hexane and collect flow through.
13. Evaporate collected samples.
14. Dissolve samples with 200 μl of 1 mg/ml PTAD in acetonitrile, vortex for 2 hours in the dark.
15. Evaporate samples.
16. Dissolve samples with 70 μl methanol, and then add 30 μl DDW. Vortex vigorously.
17. Centrifuge for 10 minutes, and transfer sample into final LC-MS vial.

### Analytical Instrumentation

Analysis was performed on an Acquity® LPC2TM system (Waters, Milford, MA, USA) equipped with a binary pump, an autosampler, a column manager oven, an atmospheric back pressure regulator (ABPR), and a make-up pump coupled to a Waters XevoTM TQ-S mass spectrometer. The whole system was controlled by MassLynxTM 4.1 software (Waters, Milford, MA, USA).

### Mass Spectrometry Instrumental Conditions

Mass spectrometric detection was carried out on Atmospheric Pressure Chemical

Ionization operating in positive mode at unit resolution (APCI+). Optimal parameters were needle corona at 4 kV, ion source temperature 150° C., probe temperature 450° C., cone gas flow 150 L/h, collision gas flow 0.15 inUmin. Argon was used as collision gas, while nitrogen was set as desolvation gas (650 L/h) (after Oberson, J. M., et al. “Quantitative analysis of vitamin D and its main metabolites in human milk by supercritical fluid chromatography coupled to tandem mass spectrometry.” Analytical and Bioanalytical Chemistry 412.2 (2020): 365-375.).

### Sterol Profiling: Extraction and Profiling

Algal material (approximately 10 mg) was homogenized (Ball Mill MM301, Retsch, Haan, Germany) in the presence of liquid nitrogen and analytes were extracted at 75° C. for 60 min with 4 mL chloroform/methanol (2:1, vol:vol; containing 0.625 mg/L epi-cholesterol as an internal standard). Extracts were kept at room temperature for at least 1 h, solvents were evaporated to dryness (EZ2-Bio, GeneVac, Ipswich, UK), and the remaining residue was saponified at 95° C. for 60 min in 2 mL 6% (w/v) KOH in methanol. Upon cooling to room temperature, 1 mL n-hexanes and 1 mL H₂O were added, and the mixture was shaken vigorously for 60 sec. Following centrifugation (1500×g for 8 min) to separate the phases, the hexane phase was transferred to a 2 mL glass vial (I do this in 1.5 ml Eppendorf tubes, as it is easy to evaporate in next step), and the aqueous phase was re-extracted with 600 uL n-hexanes as above, centrifuged as above, and the hexane phase added to the 1.5 mL Eppendorf containing the hexane phase from the first extraction. The combined hexane phases were evaporated to dryness using a gentle stream of nitrogen, 50 μL of N-methyl-N-trimethylsilyltrifluoroacetamide (MSTFA) were added to the residue, the sample was shaken vigorously for 20 sec, and the mixture was transferred to a 2 mL autosampler glass vial with a 100 μL conical glass insert (optional: after addition of MSTFA, you can incubate samples in glass vials at 70° C. for 30-45 mins and go to next step). After capping the vial, the reaction mixture was incubated at room temperature for at least 5 min.

### Analytical Instrumentation

The GC-MS system comprised a COMBI PAL autosampler (CTC Analytics), a trace GC ultra-gas chromatograph equipped with a programmable temperature vaporizing (PTV) injector, and a DSQ quadrupole mass spectrometer (Thermo Electron). GC was performed on a 30 m×0.25 mm×0.25-μm Zebron ZB-5 ms MS column (Phenomenex). The PTV split technique was performed as follows: samples were analyzed in the constant temperature splitless mode. PTV inlet temperature was set at 280° C. Analytes were separated using the following chromatographic conditions: Helium was used as carrier gas at a flow rate of 1.2 mL/min. The thermal gradient started at 170° C., was held at this temperature for 1.5 min, ramped to 280° C. at 37° C./min and then ramped to 300° C. at 1.5° C./min and held at 300° C. for 5.0 min. Eluents were fragmented in the electron impact mode with an ionization voltage of 70 eV. The reconstructed ion chromatograms and mass spectra were evaluated using Xcalibur software version 1.4 (ThermoFinnigan). Compounds were identified by comparison of their retention index and mass spectrum to those generated for authentic standards analyzed on the same instrument: α-amyrin (Apin Chemicals); β-sitosterol, β-amyrin, cholestanol, cholesterol, and stigmasterol (Sigma-Aldrich); lanosterol, cycloartenol, and campesterol (Steraloids), and 2,3-oxidosqualene (Echelon Biosciences).

(After Itkin, Maxim, et al. “TOMATO AGAMOUS-LIKE 1 is a component of the fruit ripening regulatory network.” The Plant Journal 60.6 (2009): 1081-1095.)

### EXAMPLE 1

### Identification of the Vitamin D Biosynthetic Pathway in E. huxleyi and Detection of D2 and D3 in the Algae and Production of Vitamin D2 and Vitamin D3 as Well as Other Sterols Under UVB

Using manual curations to determine accurate open reading frames, the present inventors were able to reconstruct a ‘vitamin D biosynthetic road-map’ comprising 29 genes, many of them have not been previously identified in E. huxleyi (FIG. 2). The present analysis revealed a nearly complete E. huxleyi set of genes for the processing of a squalene substrate through various sterol intermediates all the way to vitamin D2, D3 and additional phytosterols.

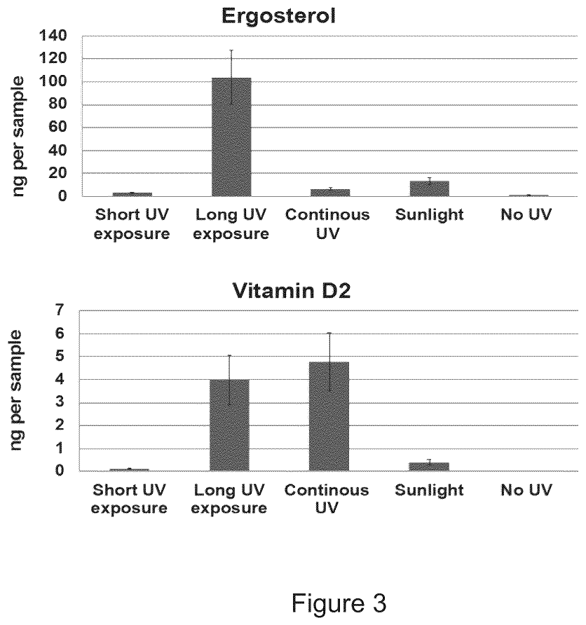

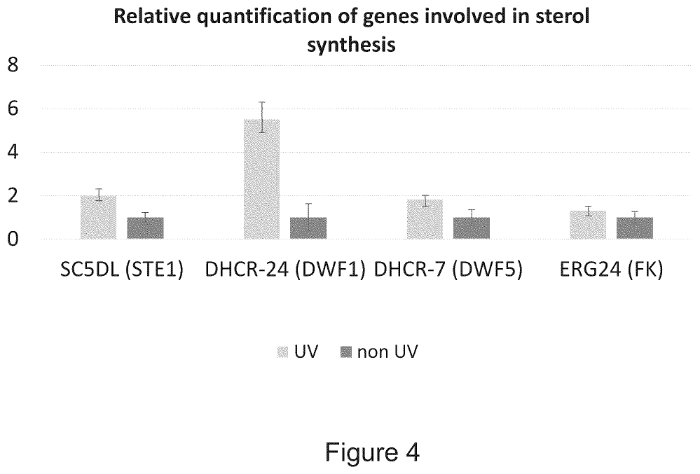

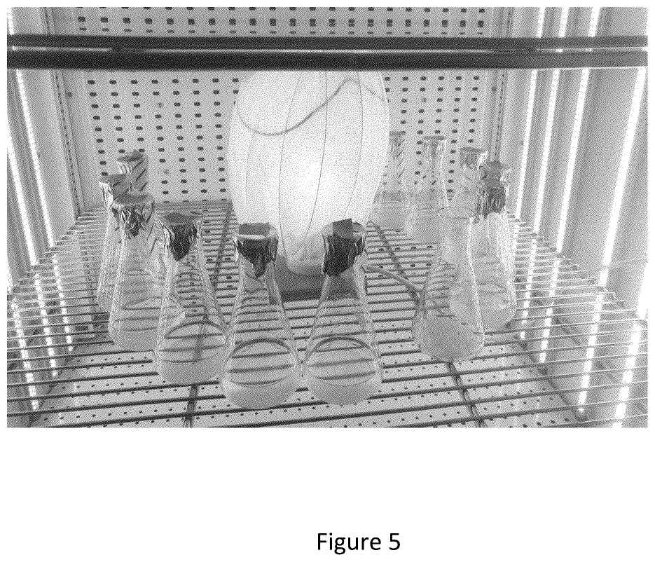

The biosynthesis of vitamin D requires UV-B in order to convert precursors into the active form. For validation of D2 and D3 production in E. huxleyi, growth was done under controlled irradiation. To achieve controlled UV-B exposure a growth chamber used for growing algal cultures was equipped with a UV-B emitting light source (FIG. 5). To directly detect vitamin D in the algal cultures, the present inventors developed a protocol for GC-MS sterols analyses. The data indicate that exposure to UVB results in detectable production of vitamin D2 and its precursor ergosterol (FIG. 3). In addition, examination via qRT-PCR of key genes involved in sterol and vitamin D production indicated that exposure to UV radiation induces their transcription (FIG. 4).

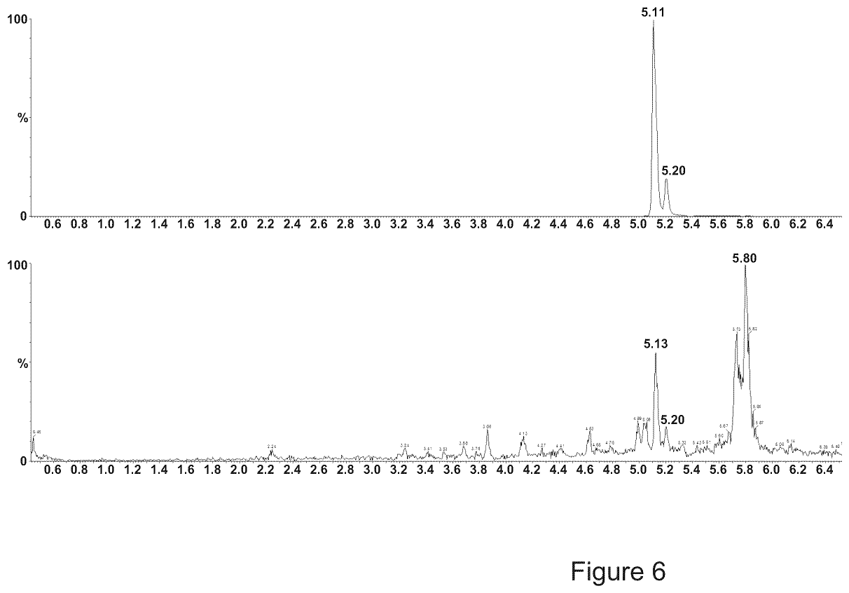

In order to test for the very first time whether E. huxleyi produces vitamin D, we conducted the following experiment. Axenic algal cultures were cultivated for two weeks under a diurnal cycle in which the light period consisted of visible light in addition to UVB in intensity of 0.07 W/m, resembling environmental values at the sea surface. Following cultivation the cultures were harvested and vitamin D was extracted using protocols developed in the lab as specified in later sections. Vitamin D analysis was performed using LC-MS on a UPC² device. FIG. 6 shows vitamin D3 detection after exposure to UV radiation by UPC². FIG. 6 shows UPC² analysis of E. huxleyi vitamin D extracts. Upper panel showing results for vitamin D3 standard. Lower panel showing extract of an algal sample with peaks matching in retention time and mass to the vitamin D3 standard. This data demonstrates for the first time the identification of vitamin D3 in the microalga E. huxleyi.

### EXAMPLE 2

### The Effect of Bacterial Co-Culture on E. huxleyi Production of Vitamin D Under UVB

To examine vitamin D production in co-cultures of E. huxleyi and the bacterium P. inhibens the present inventors cultivate in parallel, under the same conditions, multiple axenic cultures as control and multiple replicates of algal-bacterial co-cultures. All cultures are exposed to a diurnal light cycle in which the light period includes illumination with UVB. Following two weeks of cultivation, all samples are harvested and subjected to vitamin D extraction. Analysis of vitamin D is conducted via LC-MS against vitamin D standards of known identity and concentration. The analysis allows to identify various vitamin D species and their derivatives, and quantify them in the different cultures.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### (Other References are Cited in the Document)

1. Chick H. The Relation of Ultra-Violet Light to Nutrition. Lectures II. Lancet. 1932:377-384.

2. Wolf G. The Discovery of Vitamin D: The Contribution of Adolf Windaus. J Nutr. 2004; 134(6):1299-1302. doi:10.1093/jn/134.6.1299

3. Hossein-Nezhad A, Holick MF. Vitamin D for health: A global perspective. Mayo Clin Proc. 2013; 88(7):720-755. doi:10.1016/j.mayocp.2013.05.011

4. Lopez Payares G M, Ali F A. Vitamin D deficiency. 5-Minute Clin Consult Stand 2016 Twenty Fourth Ed. 2015:266-281.

5. Drummond J C, GUNTHER Er. Vitamin content of marine plankton. Nature. 1930; 126(3176):398.

6. Drummond B Y J C, Gunther E R. Observations on the Fatty Constituents of Marine Plankton: II. General Character of the Plankton Oils. J Exp Biol. 1934; 11(2):198-202.

7. Holick M F. Evolution and Function of Vitamin D. In: Reichrath J, Tilgen W, Friedrich M, eds. Vitamin D Analogs in Cancer Prevention and Therapy. Berlin, Heidelberg: Springer Berlin Heidelberg; 2003:3-28.

8. Rao D S, Raghuramulu N. Food chain as origin of vitamin D in fish. Comp Biochem Physio—A Physiol. 1996; 114(1):15-19. doi:10.1016/0300-9629(95)02024-1

9. Bouillon R, Van Cromphaut S, Carmeliet G. Intestinal calcium absorption: Molecular vitamin D mediated mechanisms. J Cell Biochem. 2003; 88(2):332-339. doi:10.1002/jcb.10360

10. Engelsen O. The relationship between ultraviolet radiation exposure and vitamin D status. Nutrients. 2010; 2(5):482-495. doi:10.3390/nu2050482

11. Balch W M. The Ecology, Biogeochemistry, and Optical Properties of Coccolithophores. Ann Rev Mar Sci. 2018; 10(1):71-98. doi:10.1146/annurev-marine-121916-063319

12. Barker S, Higgins J A, Elderfield H, McCave I N, Shepherd J G. The future of the carbon cycle: Review, calcification response, ballast and feedback on atmospheric CO2. Philos Trans R Soc A Math Phys Eng Sci. 2003; 361(1810):1977-1999. doi:10.1098/rsta.2003.1238

13. Schiebel R. Planktic foraminiferal sedimentation and the marine calcite budget. Global Biogeochem Cycles. 2002; 16(4):3-21. doi:10.1029/2001GB001459

14. Tannbutté S, Holcomb M, Ferrier-Pagès C, et al. Coral biomineralization: From the gene to the environment. J Exp Mar Bio Ecol. 2011; 408(1):58-78. doi:www(dot)doi(dot)org/10.1016/j.jembe.2011.07.026

15. Gairuso J P, Allemand D, Frankignoulle M. Photosynthesis and calcification at cellular, organismal and community levels in coral reefs: A review on interactions and control by carbonate chemistry. Am Zool. 1999; 39(1):160-183. doi:10.1093/icb/39.1.160

16. Paasche E. A review of the coccolithophorid emiliania huxleyi (prymnesiophyceae), with particular reference to growth, coccolith formation, and calcification-photosynthesis interactions. Phycologia. 2001; 40(6):503-529. doi:10.2216/i0031-8884-40-6-503.1

17. Mercer E I. The Biosynthesis of Ergosterol”. 1984; (March 1983):133-155.

18. Nanninga H J, Tyrrell T. Importance of light for the formation of algal blooms by Emiliania huxleyi. Mar Ecol Prog Ser. 1996436(1-3):195-203. doi:10.3354/meps136195

19. Tedetti M, Sempéré R. Penetration of Ultraviolet Radiation in the Marine Environment. A Review. Photochem Photobiol. 2006; 82(2):389. doi:10.1562/2005-11-09-ir-733

20. Holick M F, Holick S A, Guillard R L. On the origin and metabolism of vitamin D in the sea. Comp Endocrinol calcium Regul. 1982:85-91.

21. Brownlee C, Wheeler G L, Taylor A R. Coccolithophore biomineralization: New questions, new answers. Semin Cell Dev Biol. 2015; 46:11-16. doi:10.1016/j.semcdb.2015.10.027

22. Rost B, Riebesell U. Coccolithophores and the biological pump: responses to environmental changes. In: Thierstein H R, Young J R, eds. Coccolithophores: From Molecular Processes to Global Impact. Berlin, Heidelberg: Springer Berlin Heidelberg; 2004:99-125. doi:10.1007/978-3-662-06278-4_5

23. de Vargas C, Aubry M P, Probert I, Young J. Origin and Evolution of Coccolithophores. From Coastal Hunters to Oceanic Farmers. Elsevier Inc.; 2007. doi:10.1016/8978-012370518-1/50013-8

24. Medlin L K, Sáez AG, Young J R. A molecular clock for coccolithophores and implications for selectivity of phytoplankton extinctions across the K/T boundary. Mar Micropaleontol. 2008; 67(1):69-86. doi:www(dot)doi(dot)org/10.1016/j.marmicro.2007.08.007

25. McClelland H-LO. Carbon dioxide and coccolithophore physiology in ancient oceans VO-RT-Thesis. OP-. (WP-).

26. Stoll H M, Ziveri P. Coccolithophorid-based geochemical paleoproxies. In: Thierstein H R, Young J R, eds. Coccolithophores: From Molecular Processes to Global Impact. Berlin, Heidelberg: Springer Berlin Heidelberg; 2004:529-562. doi:10.1007/978-3-662-06278-4_20

27. Sonawane P D, Pollier J, Panda S, et al. Plant cholesterol biosynthetic pathway overlaps with phytosterol metabolism. Nat Plants. 2016; 3(December). doi:10.1038/nplants.2016.205

28. Segev E, Wyche T P, Kim K H, et al. Dynamic metabolic exchange governs a marine algal-bacterial interaction. Elife. 2016; 5(NOVEMBER2016):1-28. doi:10.7554/eLife.17473

29. Chernihovsky N, Torfstein A, Almogi-Labin A. Seasonal flux patterns of planktonic foraminifera in a deep, oligotrophic, marginal sea: Sediment trap time series from the Gulf of Aqaba, northern Red Sea. Deep Sea Res Part I Oceanogr Res Pap. 2018; 140:78-94. doi:www(dot)doi(dot)org/10.1016/j.dsr.2018.08.003

30. Villar E, Vannier T, Vernette C, et al. The Ocean Gene Atlas: exploring the biogeography of plankton genes online. Nucleic Acids Res. 2018; 46(W1):W289-W295. doi:10.1093/nar/gky376

1-24. (canceled)

25. A method of producing vitamin D, the method comprising:
a. growing an alga under controlled UV-B radiation; and
b. dehydrating the alga.

26. The method of claim 25, wherein said controlled UV-B radiation comprises: (i) long UV exposure; (ii) continuous UV exposure; or (iii) both (i) and (ii).

27. The method of claim 25, wherein said dehydrated alga comprises water in a percentage of between 1% and 5%.

28. The method of claim 25 wherein said dehydrated alga in a form of a paste.

29. The method of claim 25, wherein said alga is characterized by increased expression of at least one gene in a vitamin D biosynthetic pathway selected from the group consisting of: Delta24-sterol reductase, 7-dehydrocholesterol reductase, Delta7-sterol 5-desaturase, Deltal4-sterol reductase, and any combination thereof, under said controlled UV-B compared to a non-UV exposed control.

30. The method of claim 25, wherein said alga is characterized by at least a 3-fold increased expression of Delta24-sterol reductase, under said controlled UV-B compared to a non-UV exposed control.

31. The method of claim 25, wherein said alga is characterized by increased expression of at least one gene comprising a nucleic acid selected from the group consisting of SEQ ID NOs: 4, 15, and 31-33, and any combination thereof, under said controlled UV-B compared to a non-UV exposed control.

32. The method of claim 25, wherein said alga is characterized by at least a 3-fold increased expression of SEQ ID NO: 4 under said controlled UV-B compared to a non-UV exposed control.

33. The method of claim 25, wherein said alga is a microalga.

34. The method of claim 33, wherein said microalga is E. huxleyi.

35. The method of claim 25, wherein said UV-B is in a wavelength of between 280 nm and 315 nm.

36. The method of claim 25, wherein said UV-B is in an intensity of about 0.07 W/m.

37. The method of claim 25, further comprising determining a level of expression of a gene in a vitamin D biosynthetic pathway of said alga prior to and/or following step (b), said level being indicative of vitamin D production.

38. The method of claim 25, wherein said vitamin D comprises any one of vitamin D2, vitamin D3, and both.

39. The method of claim 25, wherein said growing is in a closed setting.

40. The method of claim 25, not comprising an extraction step.

41. The method of claim 25, wherein said growing is at a pH of between 7.2 and 9.

42. A dry biomass of an alga comprising a vitamin D in an amount of between 10 ng to 5,000 ng per gram dry weight of said dry biomass.

43. The dry biomass of claim 42, wherein said alga comprises microalga, and optionally wherein said microalga comprises E. huxleyi.

44. A method of improving nutrition of a subject in need thereof, the method comprising administering to said subject an effective amount of the dry biomass of claim 42, thereby improving the nutrition of the subject.
